(19) United States
(12) Patent Application Publication
(10) Pub. No.: US 2025/0011873 A1
(43) Pub. Date: Jan. 9, 2025

(54) ENDOCRINE TREATMENT OF HORMONE RECEPTOR POSITIVE BREAST CANCER TYPED AS HAVING A LOW RISK OF RECURRENCE

(71) Applicant: Agendia N.V., Amsterdam (NL)

(72) Inventor: Annuska Maria GLAS, Assendelft (NL)

(21) Appl. No.: 18/289,869

(22) PCT Filed: May 9, 2022
(86) PCT No.: PCT/NL2022/050251

§ 371 Date: Nov. 7, 2023

(30) Foreign Application Priority Data
May 7, 2021 (EP) 21172830.8

(51) Int. Cl. C12Q 1/6886; A61K 31/138; A61K 31/4196; A61P 35/00

(52) U.S. Cl. CPC C12Q 1/6886; A61K 31/138; A61K 31/4196; A61P 35/00; C12Q 2600/106

(57) ABSTRACT

The invention relates to endocrine therapy for use in a method of treating a hormone-receptor (HR)-positive breast cancer, that is typed as having a low risk of recurrence. The invention further relates to methods of treating an individual with HR-positive breast cancer with endocrine therapy according to the cancer's typed risk of recurrence.

This application is the U.S. National Phase of International Patent Application Number PCT/NL2022/050251 filed May 9, 2022, which claims priority from EP 21172830.8 filed May 7, 2021, each of which is incorporated herein by reference.

FIELD OF INVENTION: The invention relates to methods for treatment of cancer, especially breast cancer.

### 1 BACKGROUND

Cancer is a leading cause of death worldwide, and was responsible for nearly 10 million deaths in 2020. The most common cancer in 2020 in terms of new cases was breast cancer with 2.26 million cases and responsible for 685,000 deaths globally that year (Ferlay et al., 2021. Int J Cancer 10.1002/ijc.33588).

As in other cancers, early detection and treatment of breast cancer can effectively reduce cancer-associated mortality and significantly improve the lives of cancer patients. The overall 5-year relative survival rate of breast cancer patients is about 85%, meaning that 85% of the diagnosed breast cancer patients survive for at least five years. For patients diagnosed with early stage localized breast cancer not spread to the lymph nodes (localized) the 5-year survival rate is about 99%, while for patients diagnosed with metastasized breast cancer (distant) the 5-year survival rate is about 28% (Howlader et al. (editors). Cancer Statistics Review, 1975-2017. Table 4.13. National Cancer Institute).

Not only the stage of the disease is important for the prognosis of breast cancer patients, also the subtype classification of the breast cancer is an important prognostic factor. An example of such subtype classification is the oestrogen receptor (ER) status of a breast cancer. Patients diagnosed with ER positive early stage breast cancer tend to have better survival rates than patients with ER negative early stage breast cancer. Interestingly, this favourable prognostic effect for ER positive breast cancer tend to decrease over time, with a worse prognosis in terms of overall survival for ER positive breast cancer patients compared to ER negative patients assessed five years after diagnosis (Bentzon et al., 2008. Int J Cancer 122: 1089-1094).

Once a diagnosis of breast cancer is established and a stage of the cancer is known, an appropriate therapy can be determined. Breast cancers detected at an early stage are typically treated by surgery, often followed by radiotherapy, while metastasized cancers even if detected at an early stage, are treated systemically by chemotherapy (Maughan et al., 2010. Am Fam Physician 81: 1339-1346). Adjuvant (i.e. additional) therapy is often administered as well, depending on the type of breast cancer diagnosed, to increase survival rates. As an example, for hormone receptor (HR)-positive breast cancer, adjuvant hormone-therapy is often recommended. In addition, for a large tumour that is to be removed by surgery, adjuvant chemotherapy is often administered after removal of the primary tumour (Anampa et al., 2015. BMC Med 13, 195).

Adjuvant endocrine therapy is traditionally administered for a duration of five years and has shown to reduce the recurrence rates in woman with early-stage ER positive breast cancer substantially (Early Breast Cancer Trialist' Collaborative group, 2005. Lancet 365: 1687-1717; 2011. Lancet 378: 771-784; 2015. Lancet 386: 1341-1352). The currently used agents in endocrine therapy include anti-oestrogen drugs such as the well-known tamoxifen and aromatase inhibitors (AI). Treatment with tamoxifen for five years has shown to reduce the recurrence rate in breast cancer patients with 50% compared to placebo treated patients during the treatment period (Early Breast Cancer Trialist' Collaborative group, 2011. Lancet 378: 771-784). Aromatase inhibitors have been shown to be even more effective in postmenopausal woman resulting in about 30% less recurrences compared to tamoxifen during the treatment period of five years, but not thereafter (Early Breast Cancer Trialist' Collaborative group, 2015. Lancet 386: 1341-1352).

Extending the treatment with endocrine therapy beyond five years, is called extended endocrine therapy. Extended endocrine therapy after five years of tamoxifen with tamoxifen or an AI has been shown to improve disease free survival (DFS) in early stage breast cancer (Goss et al., 2003. Lancet 351: 1451-1467; Davies et al, 2013. Lancet 381:805-816). Although, several clinical trials report such beneficial results, the optimal length of extended endocrine therapy remains controversial (Jinih et al., 2017. The Breast J 23:694-705).

In the NSABP B42 clinical trial, investigators aim to evaluate extended adjuvant AI therapy in patients after five years of endocrine therapy. These patients are postmenopausal woman with stage I-IIIA HR positive breast cancer who remained free of breast cancer after completing years of endocrine therapy with either an AI or initial tamoxifen for up to three years followed by an AI for the remainder of five years. Although no statistically significant prolongation of disease-free survival (DFS) was reported with extended letrozole therapy compared to the placebo group, a 28% reduction in distant recurrence (DR) was observed in the letrozole arm (Mamounas et al., 2019. Lancet Oncol 20: 88-99).

Taken in mind the severe side effects of endocrine therapy, there is a need for a method to identify patient subgroups who receive greater proportional benefit from extended endocrine therapy.

### 2 SUMMARY

The invention provides endocrine therapy for use in a method of treating a hormone receptor (HR)-positive breast cancer in an individual, wherein said cancer is typed as having a low risk of recurrence and wherein said endocrine therapy is administered for more than five years.

Said endocrine therapy preferably comprises an aromatase inhibitor and/or anti-oestrogen therapy. Most preferably, said endocrine therapy administered for more than five years comprises letrozole and/or tamoxifen. Said endocrine therapy preferably has a duration of at least six years, more preferably at least seven years, more preferably at least eight years, more preferably at least nine years, most preferably at least ten years.

The cancer is preferably typed using MammaPrint. Said cancer is considered as having low risk of recurrence if it is typed as MammaPrint low risk (MP-L). Preferably, said cancer is not typed as MammaPrint ultralow risk (MP-UL).

The individual is preferably a post-menopausal woman.

The invention further provides a method of treating an individual with hormone receptor (HR)-positive breast cancer with endocrine therapy administered for more than five years if said cancer is typed as having low risk of recurrence and treating an individual with HR-positive breast cancer with endocrine therapy administered for five years or less if said cancer is typed as having high risk of recurrence.

The endocrine therapy administered for more than five years preferably comprises an aromatase inhibitor and/or anti-oestrogen therapy. Most preferably, said endocrine therapy administered for more than five years comprises letrozole and/or tamoxifen. Said endocrine therapy administered for more than five years preferably has a duration of at least six years, more preferably at least seven years, more preferably at least eight years, more preferably at least nine years, most preferably at least ten years.

The endocrine therapy administered for five years or less preferably comprises an aromatase inhibitor and/or anti-oestrogen therapy.

Said cancer is preferably typed using MammaPrint. In a preferred method of treating of the invention, said cancer is considered as having low risk of recurrence if it is typed as MammaPrint low risk (MP-L) and wherein the cancer is considered as having high risk of recurrence if it is typed as MammaPrint high risk (MP-H).

Preferably, the individual is a post-menopausal woman.

The method of treating according to the invention, wherein the individual with HR-positive breast cancer is treated with endocrine therapy administered for five years or less if said cancer is typed as having MammaPrint ultralow risk (MP-UL).

### 3 DRAWINGS

FIG. 1. Overview of the patients and samples included in the MammaPrint (MP) cohort.

FIG. 2. Distant recurrence in all B-42 patients (A), in MammaPrint Low risk patients (B), and in MammaPrint High risk patients (C).

FIG. 3. Disease-free survival for all B-42 patients (A), in MammaPrint Low risk patients (B), and in MammaPrint High risk patients (C).

FIG. 4. Time-dependent analyses of Breast Cancer Free Interval (BCFI) in MammaPrint Low risk patients (A), and in MammaPrint High risk patients (B).

### 4 DETAILED DESCRIPTION OF THE INVENTION

### 4.1 Definitions

As is used herein, the term “cancer” refers to a disease or disorder resulting from the proliferation of oncogenically transformed cells.

As is used herein, the term “breast cancer” is any cancer originating from cells of the breasts.

As is used herein, the term “tissue sample” refers to any tissue that can be completely or partly obtained from an individual by various means including, for example, biopsy such as needle biopsy and surgery. The term comprises any tissue sample comprising breast cancer cells from an individual, or suspected to comprise breast cancer cells from an individual, such as a tumour or liquid biopsy. Preferably, at least 5% of the tissue sample consists of breast cancer cells. More preferably at least 10%, 20% or 30% of the tissue sample consists of breast cancer cells. The term also comprises any tissue sample that may comprise gene expression products from breast cancer cells from an individual, such as blood, thrombocytes and erythrocytes.

As is used herein, the term “typing of a sample” refers to the classification of a sample based on characterized features. In this invention typing includes the characterisation of expression levels of genes in a sample assisting in the prediction of the risk of recurrence of a breast cancer.

As used herein, the term “recurrence” refers to the presence of cancer cells after a cancer-free period in which the cancer was undetectable, usually after treatment. Cancer recurrence can be either locally, regionally or distantly.

As used herein, the term “local recurrence”, refers to detection of cancer cells in the same place or near the same place as before the cancer-free period. In the case of breast cancer, local recurrence is referred to as recurrence in the breast area where it was originally detected.

As used herein, the term “regional recurrence” refers to detection of the cancer in the lymph nodes near the place where it was detected before the cancer-free period. In the case of breast cancer, regional recurrence is referred to as recurrence in the lymph nodes of the armpit or collarbone area.

As used herein, the term “distant recurrence”, also called “metastatic recurrence”, refers to detection of the cancer in another body part as where it was detected before the cancer-free period. In the case of breast cancer, distant recurrence can be detected in organs such as bones, liver, brain and lungs, but also recurrence in the opposite breast is called distant recurrence.

As is used herein, the term “risk of recurrence” refers to the probability or likelihood of breast cancer recurrence.

As is used herein, the term “individual” refers to a human. Said individual preferably is a woman. Said individual preferably is a postmenopausal woman.

As is used herein, the term “postmenopausal” refers to a woman having age 56 or older with no spontaneous menses for at least 12 months or an individual having age 55 or younger with no spontaneous menses for at least 12 months with a documented oestradiol level in the postmenopausal range according to local institutional/laboratory standards, or a prior documented bilateral oophorectomy.

As is used herein, the term “adjuvant therapy” refers to treatment given following a primary treatment such as surgery. An aim of adjuvant therapy is, for example, to remove cancer cells that remained after primary treatment and/or to reduce the chance of recurrence of cancer cells. Adjuvant therapy in breast cancer, in addition to surgery, involves treatment including one or more of chemotherapy, radiotherapy, immune therapy, targeted therapy and hormone therapy.

As is used herein, the term “endocrine therapy” also referred to as hormonal therapy or hormone therapy, refers to treatment of hormone-sensitive cancers by administering agents that interfere with hormone pathways. Hormone-sensitive cancers rely on hormones to grow. In breast cancer, endocrine therapy includes the use of selective oestrogen receptor modulators (SERMs) such as tamoxifen, the use of selective oestrogen receptor down-regulator (SERDs) such as fulvestrant, the use of aromatase inhibitors (AI) such as letrozole, the use of ovarian suppression drugs such as goserelin, or a combination thereof.

As used herein, the term “anti-oestrogen therapy” refers to endocrine treatment with selective oestrogen receptor modulators (SERMs) or with selective oestrogen receptor down-regulator (SERDs).

As is used herein, the term “oestrogen-receptor (ER) positive breast cancer” refers to a breast cancer that detectably expresses oestrogen receptor (ER). ER status may be determined, for example, by IHC and/or by TargetPrint® analysis as previously reported (Roepman et al, 2009. Clin Cancer Res 15: 7004-7011).

As is used herein, the term “progesterone-receptor (PR) positive breast cancer” refers to a breast cancer that detectably expresses progesterone receptor (PR). PR status may be determined, for example, by THC and/or by TargetPrint® analysis as previously reported (Roepman et al., 2009. Clin Cancer Res 15: 7004-7011).

As is used herein, the term “hormone-receptor (HR) positive breast cancer” refers to a breast cancer that detectably expresses oestrogen receptor (ER) and/or progesterone receptor (PR).

As is used herein, the term “human epidermal growth factor receptor 2 (HER2) negative breast cancer” refers to a breast cancer that does not detectably express human epidermal growth factor receptor 2 (HER2). HER2 is also termed v-erb-b2 avian erythroblastic leukaemia viral oncogene homolog 2 (ERBB2) or NEU. HER2 status may be determined, for example, by immunohistochemistry, chromogenic or fluorescence in situ hybridization, and/or by TargetPrint® analysis as previously reported (Roepman et al, 2009. Clin Cancer Res 15: 7004-7011).

As is used herein, the term “RNA” refers to ribonucleic acid. The term RNA includes reference to messenger RNA (mRNA).

As is used herein, the term “isolating RNA” refers to the extraction and purification of RNA from a biological sample. The term “isolating” refers to the removal of other components, such as proteins and DNA, at least to some extent. As is used herein, the term “gene expression level” refers to a quantifiable level of expression of a gene of interest. A gene's expression level is often inferred by measuring a level of a gene product, such as mRNA or protein, of that gene in a sample. Said gene expression level can be determined relatively, in relation to the expression levels of other genes, such as household genes or normalization genes as described in, for example, international patent application WO2008039071; or absolutely, for example by comparing a determined level of expression to a calibration curve of the expression product of the gene.

As is used herein, the term “expression profile” refers to the expression levels of multiple genes in a sample. An expression profile can be obtained, for example, by analysing the hybridisation pattern of a sample on a microarray, or by techniques such as RNA-sequencing or multiplex qPCR.

As is used herein, the term “marker gene” refers to a gene whose sequence or expression level, alone or in combination with other genes, is correlated with an effect, in this application a probability of a positive or negative response following auxiliary immune therapy.

As is used herein, the term “microarray gene expression analysis” refers to the analysis of gene expression levels of a predefined gene set through hybridization. Microarrays, also known as chips, are microscopic slides containing microscopic spots of nucleic acid molecules from a specific gene. The nucleic acid molecules attached to the microarray act as probes for a nucleic acid molecule such as RNA or copy-DNA (cDNA) molecule, from an experimental sample. These cDNA molecules may be labelled, for example fluorescently labelled, prior to hybridization to the microarray.

The term “hybridization”, as is used herein, refers to the binding of a nucleic acid molecule such as RNA or cDNA molecule to a (partially) complementary nucleic acid probe on the microarray. Hybridization of a labelled nucleic acid molecule may result in a signal, for example a fluorescent signal, that can be detected and quantified, yielding information about the abundance of the labelled nucleic acid molecule in the experimental sample. Microarray analysis allows for the simultaneous detection of gene expression levels of a large number of genes.

As is used herein, the term “amplification” refers to an increase in the number of copies of a particular DNA fragment through replication using a least one primer and a DNA polymerase. Known amplification methods include polymerase chain reaction (PCR) and isothermal amplification including, for example, helicase-dependent amplification (HDA) (Vincent et al., 2004. EMBO Rep 5: 795-800), loop-mediated amplification (LAMP) (Notomi et al., 2000. Nucleic Acids Res 28: E63), nucleic acid sequences-based amplification (NASBA) (Guatelli et al., 1990. Proc Natl Acad Sci USA 87: 1874-1878), rolling circle amplification (Ali et al., 2014. Chem Soc Rev 43: 3324-3341), strand-displacement amplification (SDA) (Walker et al., 1992. Nucleic Acids Res 20: 1691-6) and recombinase polymerase amplification (RPA) (Piepenburg et al., 2006. PLoS Biology 4: e204).

As is used herein, the term “RNA-Seq”, also termed RNA-sequencing, refers to a sequencing technique, such as a high-throughput sequencing technique, preferably using next-generation sequencing (NGS), to characterize the quantity and/or sequence of a nucleic acid molecule such as RNA in a sample. RNA-Seq can be used for gene expression analysis.

### 4.2 Typing of an Individual's Risk of Cancer Recurrence

In this invention, an individual with breast cancer can be typed using several different methods known in the art. Said individual with breast cancer can be an individual diagnosed with breast cancer or likely to be diagnosed with breast cancer. Said individual with breast cancer is an individual suffering from breast cancer or likely to suffer from breast cancer. Typing of an individual's risk of cancer recurrence is preferably performed on a tissue sample, comprising breast cancer cells or breast cancer derived nucleic acids, from said individual. Typing of said sample is preferably performed by isolating RNA molecules from said tissue sample and determining a gene expression profile.

### 4.2.1. Sample Collection and Pre-Processing

The tissue sample may be obtained from any individual with breast cancer. The individual preferably is a woman. The tissue sample may comprise any tissue sample comprising breast cancer cells or breast cancer derived nucleic acids from said individual such as a tumour or liquid biopsy.

The term “biopsy” refers to a biopsy derived from a primary breast cancer, such as a needle biopsy.

Said tumour biopsy can be obtained by in numerous ways, as is known to a person skilled in the art. Preferably, the biopsy is obtained using needle biopsy or surgical biopsy. During needle biopsy, cancer cells are extracted from the breast cancer using a needle. During surgical biopsy, cells are extracted from the breast cancer after making an incision in the skin. In individuals with breast cancer, surgical biopsy is often part of a primary treatment, in which the cancer, or parts thereof, is removed from the body. It is explicitly stated that the act of removing a breast cancer, or a part of a breast cancer, from an individual is not part of this invention.

The term “liquid biopsy” refers to a biopsy obtained from a bodily fluid comprising circulating breast cancer cells or cells that have absorbed nucleic acids derived therefrom such as educated thrombocytes and/or erythrocytes (Best et al, 2015. Cancer Cell 28: 666-676; Heinhuis et al., 2020. Cancers 12: 1372).

Several body fluids can potentially contain circulating breast tumour cells such as blood, plasma, serum, lymphatic fluid, saliva, faeces, urine and cerebrospinal fluid. Preferably, blood or plasma is preferably used as bodily fluid to provide a liquid biopsy of breast cancer.

The tissue sample may be collected in any clinically acceptable manner, but is preferably collected and conserved upon isolation such as to preserve at least RNA. RNA can be obtained from a tissue sample immediately upon harvesting, or from a conserved tissue sample. A tissue sample can be conserved by fixation e.g. in formalin and/or by treating the tissue sample with an RNase inhibitor, such as RNasin (Promega) and RNasecure (Invitrogen), or an RNA stabilisation agent, such as RNAlater (Invitrogen). Preferred conservation methods of tissue samples include fresh frozen (FF) conservation, for example in dry ice or in liquid nitrogen, and formalin-fixed paraffin-embedded (FFPE) conservation.

RNA can be isolated from a tissue sample by methods known in the art. There are three main categories of RNA extraction techniques known to date: organic extraction involving a chaotropic agent such as guanidinium thiocyanate or guanidinium isothiocyanate, followed by, for example, phenol-chloroform extraction, silica-based column techniques (e.g. RNeasy Kit by Qiagen) and magnetic beads-based techniques (e.g. Dynabeads by Invitrogen). A preferred method involves guanidinium thiocyanate-extraction such as, e.g. TRlzol® Kit by Invitrogen.

### 4.2.2. Determining Expression Levels of Marker Genes

To type an individual's risk of recurrence, either locally, regionally or distantly, an expression level of one or more marker genes can be determined. A marker gene is a gene whose sequence or expression level, alone or in combination with other genes, is correlated with a specific effect, in this application, a risk of recurrence.

Preferably in this invention the expression level of at least 5 genes indicated in Table 1 is determined, more preferred at least 6 genes, more preferred at least 7 genes, more preferred at least 8 genes, more preferred at least 9 genes, more preferred at least 10 genes, more preferred at least 20 genes, more preferred at least 30 genes, more preferred at least 40 genes, more preferred at least 50 genes, more preferred at least 60 genes, more preferred at least 70 genes indicated in Table 1, such as all 231 genes listed in Table 1. A preferred set of genes comprises both positively correlated genes as well as negatively correlated genes, as indicated in Table 1, whereby said correlation is to a good prognosis signature.

A further preferred method of the invention for typing an individual's risk of recurrence involves the use of at least 5 genes indicated in Table 2, more preferred at least 6 genes, more preferred at least 7 genes, more preferred at least 8 genes, more preferred at least 9 genes, more preferred at least 10 genes, more preferred at least 20 genes, more preferred at least 30 genes, more preferred at least 40 genes, more preferred at least 50 genes, more preferred at least 60 genes, more preferred all 70 genes indicated in Table 2.

It is noted that some probes hybridize to the same genes indicated in Table 2, such as three probes which are now known to hybridize to expression products of the Diaphanous Related Formin 3 (DIAPH3; ENSG00000139734) gene. A reference to different genes listed in Table 2 includes reference to different probes hybridizing to the same gene listed in Table 2. Hence, the term “at least five genes of Table 2” provides reference to both 5 different genes listed in Table 2 as well as 5 different probes listed in Table 2.

A preferred set of genes for typing an individual's risk of recurrence involves a subset of 70 genes, which are indicated in Table 2 and for which preferred probes are provided in Table 2. This subset of 70 genes, or 70 gene signature, is known for its use in the MammaPrint® test (also termed “Amsterdam gene signature test” or MP) that uses the expression levels of said 70 genes (Table 2) to assess a patient's risk of developing distant metastases within 5 years after diagnosis (i.e. risk of recurrence).

The determination of an expression level of one or more marker genes may be accomplished by any means known in the art such as Northern blotting, quantitative (qPCR), microarray analysis or RNA-seq. Preferably, the expression levels of multiple marker genes are assessed simultaneously, for example by multiplex qPCR, microarray analysis or RNA-seq.

Microarray analysis involves the use of selected probes that are immobilized on a solid surface, an array. Said probes are able to hybridize to gene expression products such as mRNA, or derivates thereof such as cDNA. The probes are exposed to labeled sample gene expression products, or labelled derivates thereof, hybridized, washed, where after the abundance of gene expression products or derivates thereof in the sample that are complementary to a probe is determined by determining the amount of label that remains associated to a probe. The probes on a microarray may comprise DNA sequences, RNA sequences, or copolymer sequences of DNA and RNA. The probes may also comprise DNA and/or RNA analogues such as, for example, nucleotide analogues or peptide nucleic acid molecules (PNA), or combinations thereof. The sequences of the probes may be full or partial fragments of genomic DNA. The sequences may also be in vitro synthesized nucleotide sequences, such as synthetic oligonucleotide sequences.

In the context of the invention, a probe preferably is specific for a gene expression product of a gene as listed in Table 1. A probe is specific when it comprises a continuous stretch of nucleotides that are completely complementary, over the whole length, to a nucleotide sequence of a gene expression product, or a cDNA product thereof. A probe can also be specific when it comprises a continuous stretch of nucleotides that are partially complementary to a nucleotide sequence of a gene expression product of said gene, or a cDNA product thereof. Partially means that a maximum of 5 nucleotides, more preferable 4 nucleotides, more preferable 3 nucleotides, more preferable 2 nucleotides and most preferable one nucleotide differs from the corresponding nucleotide sequence of a gene expression product of said gene. The term complementary is known in the art and refers to a sequence that is related by base-pairing rules to the sequence that is to be detected. It is preferred that the sequence of the probe is carefully designed to minimize nonspecific hybridization to said probe.

The specificity of probe is further determined by the hybridization and/or washing conditions. The hybridization and/or washing conditions are preferably stringent, which are determined by inter alia the temperature and salt concentration of the hybridization and washing conditions, as is known to a person skilled in the art. An increased stringency will substantially reduce non-specific hybridization to a probe, while specific hybridization is not substantially reduced. Stringent conditions include, for example, washing steps for five minutes at room temperature 0.1×sodium chloride-sodium citrate buffer (SSC)/0.005% Triton X-102. More stringent conditions include washing steps at elevated temperatures, such as 37° Celsius, 45° Celsius, or 65° Celsius, either or not combined with a reduction in ionic strength of the buffer to 0.05×SSC or even 0.01×SSC, as is known to a skilled person.

It is preferred that the probe is, or mimics, a single stranded nucleic acid molecule. The length of a probe can vary between 15 bases and several kilo bases, and is preferably between 20 bases and 1 kilobase, more preferred between 40 and 100 bases, and most preferred about 60 nucleotides. A most preferred probe comprises about 60 nucleotides. Said probe is preferably identical over the whole length to a nucleotide sequence of a gene expression product of a gene, or a cDNA product thereof. In a method of the invention, probes comprising probe sequences as indicated in Table 1 can be employed.

To determine an RNA expression level by micro arraying, gene expression products in the sample are preferably labeled, either directly or indirectly, and contacted with probes on the array under conditions that favor duplex formation between a probe and a complementary molecule in the labeled gene expression product sample. The amount of label that remains associated with a probe after washing of the microarray can be determined and is used as a measure for the gene expression level of a nucleic acid molecule that is complementary to said probe.

Image acquisition and data analysis can subsequently be performed to produce an image of the surface of the hybridized array. For this, the array may be dried and placed into a laser scanner to determine the amount of labeled sample that is bound to a probe at a predetermined spot. Laser excitation will yield an emission with characteristic spectra that is indicative of the labelled sample that is hybridized to a probe molecule.

An array preferably comprises multiple spots encompassing a specific probe. A probe preferably is present in duplicate, in triplicate, in quadruplicate, in quintuplicate, in sextuplicate or in octuplicate on an array. The multiple spots preferably are at randomized opposition on an array to minimize bias. The amount of label that remains associated with the probe at each spot may be averaged, where after the averaged level can be used as a measure for the gene expression level of a nucleic acid molecule that is complementary to said probe. In addition, a gene product may be hybridized to two or more different probes that are specific for that gene product.

The determined RNA expression level can be normalized for differences in the total amounts of nucleic acid expression products between two separate samples by comparing the level of expression of one or more genes that are presumed not to differ in expression level between samples such as glyceraldehyde-3-phosphate-dehydro-genase, β-actin, and ubiquitin. Conventional methods for normalization of array data include global analysis, which is based on the assumption that the majority of genetic markers on an array are not differentially expressed between samples (Yang et al, 2002. Nucl Acids Res 30:15). Alternatively, the array may comprise specific probes that are used for normalization. These probes preferably detect RNA products from housekeeping genes such as glyceraldehyde-3-phosphate dehydrogenase and 18S rRNA levels, of which the RNA level is thought to be constant in a given cell and independent from the developmental stage or prognosis of said cell.

Another preferred method for determining RNA expression levels is by sequencing, preferably next-generation sequencing (NGS), of RNA samples, with or without prior amplification of the RNA expression products. High throughput sequencing techniques for sequencing RNA, or RNA-seq, have been developed. NGS platforms, including Illumina® sequencing; Roche 454 pyrosequencing®, ion torrent and ion proton sequencing, and ABI SOLiD® sequencing, allow sequencing of fragments of DNA in parallel. Bioinformatics analyses are used to piece together these fragments by mapping the individual reads. Each base is sequenced multiple times, providing high depth to deliver accurate data and an insight into unexpected DNA variation. NGS can be used to sequence a complete exome including all or small numbers of individual genes.

Pyrosequencing detects the release of inorganic pyrophosphate (PPi) as particular nucleotides are incorporated into the nascent strand (Ronaghi et al, 1996. Analytical Biochemistry 242:84-9; Ronaghi, 2001. Genome Res 11: 3-11; Ronaghi et. al., 1998. Science 281: 363; U.S. Pat. Nos. 6,210,891; 6,258,568; and 6,274,320, which are all incorporated herein by reference. In pyrosequencing, released PPi can be detected by being immediately conversion to adenosine triphosphate (ATP) by ATP sulfurylase, and the level of ATP generated is detected via luciferase-produced photons.

NGS also includes so called third generation sequencing platforms, for example nanopore sequencing on an Oxford Nanopore Technologies platform, and single-molecule real-time sequencing (SMRT sequencing) on a PacBio platform, with or without prior amplification of the RNA expression products.

Further high throughput sequencing techniques include, for example, sequencing-by-synthesis. Sequencing-by-synthesis or cycle sequencing can be accomplished by stepwise addition of nucleotides containing, for example, a cleavable or photobleachable dye label as described, for example, in U.S. Pat. Nos. 7,427,673; 7,414,116; WO 04/018497; WO 91/06678; WO 07/123744; and U.S. Pat. No. 7,057,026, all of which are incorporated herein by reference.

Sequencing techniques also include sequencing by ligation techniques. Such techniques use DNA ligase to incorporate oligonucleotides and identify the incorporation of such oligonucleotides and are inter alia described in U.S. Pat. No. 6,969,488; 6,172,218; and 6,306,597. Other sequencing techniques include, for example, fluorescent in situ sequencing (FISSEQ), and Massively Parallel Signature Sequencing (MPSS).

Sequencing techniques can be performed by directly sequencing RNA, or by sequencing a RNA-to-cDNA converted nucleic acid library. Most protocols for sequencing RNA samples employ a sample preparation method that converts the RNA in the sample into a double-stranded cDNA format prior to sequencing. Conversion of RNA into cDNA and/or cRNA using a reverse-transcriptase enzyme such as M-MLV reverse-transcriptase from Moloney murine leukemia virus, or AMV reverse-transcriptase from avian myeloblastosis virus, is known to a person skilled in the art.

Quantitative PCR (qPCR), or real-time PCR (RT-PCR), is a technique which is used to amplify and simultaneously quantify a template nucleic acid molecule such as an RNA. The detection of the amplification product can in principle be accomplished by any suitable method known in the art. The amplified products may be directly stained or labelled with radioactive labels, antibodies, luminescent dyes, fluorescent dyes, or enzyme reagents. Direct DNA stains include for example intercalating dyes such as acridine orange, ethidium bromide, ethidium monoazide or Hoechst dyes. These intercalating dyes are non-specific and bind to all double stranded DNA in the PCR. An increase in DNA products during amplification, results in an increased fluorescence intensity being measured. Another direct DNA detection method includes the use of sequence specific DNA probes consisting of a fluorescent reporter and quencher. Upon binding of the probe to its complementary sequence, polymerases of the PCR break the proximity of the reporter and the quencher, resulting in the emission of fluorescence. Commonly used reporter dyes include FAM (Applied Biosystems), HEX (Applied Biosystems), ROX (Applied Biosystems), YAK (ELITech Group) or VIC (Life Technologies) and commonly used quenchers include TAMRA (Applied Biosystems), BHQ (Biosearch Technologies) and ZEN (Integrated DNA Technologies). Alternatively, the amplified product may be detected by incorporation of labelled dNTP bases into the synthesized DNA fragments. Detection labels which may be associated with nucleotide bases include, for example, fluorescein, cyanine dye and BrdUrd.

For the simultaneous detection of multiple nucleic acid gene expression products, a multiplex qPCR can be used. In multiplex qPCRs, two or more template nucleic acid molecules are amplified and quantified in the same reaction. A commonly used method of achieving the simultaneously detection of multiple targets, is by using probes with different fluorescent dyes to distinguish distinct nucleic acid targets.

It is preferred in methods of the invention that genes are selected for normalization of the raw data. Preferred genes are genes of which the RNA expression levels are largely constant between individual tissue samples comprising breast cancer cells from one individual, and between tissue samples comprising breast cancer cells from different individuals. It will be clear to a skilled artisan that the RNA levels of said set of normalization genes preferably allow normalization over the whole range of RNA levels. An example of such a set of normalization genes is provided in WO 2008/039071, which is hereby incorporated by reference.

Normalization methods that may be employed include, for example, mean correction, linear combination of factors, Bayesian methods and non-linear normalization methods such quantile normalization. Preferred methods include non-parametric regression methods such as locally estimated scatterplot smoothing (LOESS; Jacoby, 2000. Electoral Studies 19: 577-613) and locally weighted scatterplot smoothing (LOWESS; Cleveland et al., 1988. J American Statistical Association 83: 596-610).

### 4.2.3 Determining of an Individual's Risk of Recurrence

Typing of a tissue sample to predict an individual's risk of recurrence can be performed in various ways. In one method, the difference or similarity between a sample's expression profile and a previously established reference expression profile is determined. The sample's expression profile is composed of the expression levels of a set of marker genes in said sample. The reference expression profile is composed of the average expression levels of the same set of marker genes in samples from a reference group. The reference group may comprise a single individual. Preferably the reference group comprises the average expression levels of at least 10, 25, 50, 100, 200 or 300 individuals. The reference group may include individuals with both high and low risk of recurrence. The reference group may also include individuals that all have a high risk of recurrence (i.e. high risk reference group) or the reference group can also be composed of individuals that all have a low risk of recurrence (i.e. low risk reference group). Alternatively, an expression profile of an individual can also be typed by comparing the individual's reference profile to multiple reference profiles. For example, the individual's expression profile can be compared to both reference profiles identified above (i.e. the high risk reference group and the low risk reference group). If the expression profile of the individual's sample is substantially more similar to the high risk reference group, when compared to the low risk reference group, it will be predicted as high risk.

The difference or similarity between an expression profile and one or more reference profiles can be determined by determining a correlation of the expression levels of marker genes in the profiles. For example, one can determine whether the expression levels of a subset of marker genes in a tissue sample correlate to the expression levels of the same subset of marker genes in a reference profile. This correlation can be numerically expressed using a correlation coefficient. Several correlation coefficients can be used. Preferred methods are parametric methods which assume a normal distribution of the data. One of these methods is the Pearson product-moment correlation coefficient, which is obtained by dividing the covariance of the two variables by the product of their standard deviations.

Said correlations between the expression levels of marker genes in the individual's sample and the reference group, can be used to produce an overall similarity score for the set of marker genes used. A similarity score is a measure of the average correlation of gene expression levels of a set of genes in a tissue sample from an individual diagnosed with breast cancer and a reference profile. Said similarity score can, but does not need to be, a numerical value between +1, indicative of a high correlation between the gene expression level of the set of genes in a sample of said individual and said reference profile, and −1, which is indicative of an inverse correlation. A threshold can be used to differentiate between samples having a low risk of recurrence, and samples having a high risk of recurrence. Said threshold is an arbitrary value that allows for discrimination between samples from individuals with a low risk of recurrence, and samples of individuals with a high risk of recurrence. If a similarity threshold value is employed, it is preferably set at a value at which an acceptable number of patients with high risk of recurrence would score as false negatives, and an acceptable number of patients with low risk of recurrence would score as false positives. A preferred threshold is 0, meaning that the similarity score is neither indicative of a high correlation between the gene expression level of the set of genes in a sample of said individual and a reference profile from patients having a low risk of recurrence, nor between the gene expression level of the set of genes in a sample of said individual and a reference profile from patients having a high risk of recurrence.

Additionally, a threshold can be used to differentiate within the samples having a low risk of recurrence between samples having an ultralow risk of recurrence and samples having a low but not ultralow risk Said threshold is an arbitrary value that allows for discrimination between samples from individuals with an ultralow risk of recurrence, and samples of individuals with a low but not ultralow risk of recurrence. If a similarity threshold value is employed, it is preferably set at a value at which an acceptable number of patients with ultra-low risk of recurrence would score as false negatives, and an acceptable number of patients with low not ultralow risk of recurrence would score as false positives.

### 4.3 Predicting a Response to Endocrine Therapy

A method of typing of the invention may further comprise determining a response to endocrine therapy.

MP is intended to classify an individual with breast cancer as having a good prognosis having no distant metastases within five years of initial diagnosis (low risk outcome), or as having a poor prognosis having distant metastases within five years of initial diagnosis (high risk outcome). MP was shown to successfully predict recurrence free survival and overall survival in retrospective and prospective studies (van de Vijver et al., 2002. N Engl J Med 347: 1999-2009; van't Veer et al., 2002. Nature 415: 530-536; Drukker et al., 2013. Int J Cancer 133: 929-936; Cardoso et al., 2016. N Engl. J Med 375: 717-729).

Using the MP test a patient tumor sample can be classified into a High Risk or Low Risk of recurrence group by a 0.00 threshold in the MammaPrint index. If the MammaPrint index is >0, the sample is classified as low risk; when the index is ≤0, then the sample is classified as high risk A sample classified as ultralow was determined in a group of untreated patients where none of the patients developed a recurrence within 5 years (Delahaye et al., 2013. BCRT 10: 801-811). A patient tumor sample can be classified as ultralow risk by MammaPrint if it has a MP index above a threshold, for example above 0.355.

It is now surprisingly found that MP may also be used to predict a response to endocrine therapy of a HR positive breast cancer patient More specifically, an individual with a HR positive breast cancer and typed as having MammaPrint low risk (MP L), thus with a good prognosis, is likely to provide a favorable response upon endocrine treatment. This favorable response was observed in terms of an improvement of different measures namely distant recurrence, disease free survival and breast cancer free interval.

Furthermore, an individual with a HR positive breast cancer and typed as having MammaPrint low, but not ultralow risk (MP LNUL) is likely to provide a favorable response upon endocrine treatment. This favorable response was observed in terms of an improvement of different measures namely distant recurrence, disease free survival and breast cancer free interval.

### 4.4 Methods of Treating an Individual with Breast Cancer

A method of treatment of breast cancer is usually determined based on the grade of the cancer, the stage of the cancer, the cancer's molecular subtype, or any combination thereof. The most common breast cancer molecular subtypes include breast cancers expressing a molecular target such as ER, progesterone receptor (PR) or HER2, and are classified as ER positive, HER2 positive, or triple negative, a breast cancer that lacks the expression of all three molecular targets.

For a non-metastatic breast cancer, primary treatment involves local treatment including surgery and often adjuvant post-operative radiotherapy. Surgery aims at the complete removal of the cancer tissue. In some instances, one or more of the axillary lymph nodes is removed as well.

There are two main types of breast cancer surgery, namely breast-conserving surgery and mastectomy. breast-conserving surgery is surgery in which cancer tissue and some surrounding normal tissue is removed from the body, but this removal only involves a part of the breast. Mastectomy is surgery where the entire breast is removed, including all of the breast tissue and sometimes nearby tissues as well. Mastectomy is called double mastectomy, when both breasts are removed.

Treatment of a nonmetastatic breast cancer may also involve systemic treatment depending on the molecular subtype of the breast cancer and is administered in addition to surgery. For hormone receptor positive (HR-positive; meaning ER and PR positive) breast cancer systemic treatment comprises endocrine therapy with or without chemotherapy. For HER2-positive breast cancer systemic therapy comprises chemotherapy combined with HER2-targeting therapy, by for example HER2-directed antibodies. For triple negative breast cancer, adjuvant therapy is mainly limited to chemotherapy.

The invention provides a method of treating an individual with HR-positive breast cancer with endocrine therapy administered for more than five years if said cancer is typed as having low risk of recurrence and treating an individual with HR-positive breast cancer with endocrine therapy administered for five years or less if said cancer is typed as having high risk of recurrence.

The invention provides a use of endocrine therapy for the treatment of a HR-positive breast cancer in an individual, wherein said cancer is typed as having low risk of recurrence and wherein said endocrine therapy administered for more than five years.

The invention provides endocrine therapy for use in the treatment of a HR-positive breast cancer in an individual, wherein said cancer is typed as having low risk of recurrence and wherein said endocrine therapy administered for more than five years.

The invention provides a use of endocrine therapy for the preparation of a medicament for the treatment of a HR-positive breast cancer in an individual, wherein said cancer is typed as having low risk of recurrence and wherein said endocrine therapy administered for more than five years.

Agents used in endocrine therapy of individuals diagnosed with breast cancer can include: selective oestrogen receptor modulators (SERMs) such as acolbifene (Endoceutics), afimoxifene (BHR Pharma, Atossa Therapeutics), arzoxifene (Eli Lilly and company), bazedoxifene (Pfizer), clomifene (Sanofi), droloxifene (Pfizer), endoxifen (Atossa Therapeutics), lasofoxifene (Pfizer), ospemifene (Osphena), pipindoxifene (LEAPChem), raloxifene (Daiichi Sankyo), tamoxifen (Rosemont Pharmaceuticals) and toremifene (Orion Corporation); selective oestrogen receptor down-regulators (SERDs) such as amcenestrant (also called SAR439859, Sanofi), AZD-9496 (AstraZeneca), AZD-9833 (AstraZeneca), brilanestrant (also called ARN-810 or GDC-0810, Genentech), D-0502 (InventisBio), elacestrant (also called RAD-1901 or ER-306323, Radius Pharmaceuticals), etacstil (also called GW-5638 or DPC974, Bristol Myers Squibb), fulvestrant (AstraZeneca), giredestrant (also called GDC-9545, Roche), LSZ102 (Novartis), LY3484356 (Eli Lilly and company), rintodestrant (G1 Therapeutics), SHR9549 (Jiangsu HengRui Medicine) and ZN-c5 (Zeno Alpha); aromatase inhibitors (AI) such as 1,4,6-androstatriene-3,17-dione (ATD), 4-androstene-3,6,17-trione (4-AT), aminoglutethimide (Novartis), anastrozole (AstraZeneca), exemestane (Pfizer), fadrozole (Novartis), formestane (Novartis), letrozole (Novartis), testolactone (Bristol Myers Squibb) and vorozole (Janssen Pharmaceutica); and gonadotropin-releasing hormone agonists to induce ovarian suppression such as leuprolide acetate (also called leuprolin, Sanofi and Astellas) and goserelin (AstraZeneca)

Megestrol, a man-made version of progesterone, is another endocrine agent that can be administered to breast cancer patients to treat the loss of appetite and severe weight loss.

In this invention, preferably, the endocrine therapy includes treatment with a SERM, SERD and an aromatase inhibitor. Said SERM preferably is raloxifene or tamoxifen. Said SERM is most preferably tamoxifen. Raloxifene is preferably administered orally, preferably daily. Raloxifene is preferably administered at dosages of 20-600 mg/day, more preferably 60-120 mg/day. Tamoxifen is preferably administered orally, preferably daily. Tamoxifen is preferably administered at dosages of 10-100 mg/day, more preferably 20-40 mg/day. Said SERD preferably is fulvestrant Fulvestrant is preferably administered intramuscular, preferably every two weeks or monthly. Fulvestrant is preferably administered at dosages of 100-1000 mg, more preferably 250-500 mg. Said aromatase inhibitor is preferably administered orally, preferably daily. In this invention, the aromatase inhibitor is preferably letrozole (preferably 0.5-15 mg/day, more preferably 2.5-7.5 mg/day), anastrozole (preferably 0.1-10 mg/day, more preferably 0.5-1 mg/day) or exemestane (preferably 0.5-600 mg/day, more preferably 25-50 mg/day).

Note that aromatase inhibitors are only effective in postmenopausal women. Physicians sometimes decide to induce the menopause in women before starting endocrine treatment Menopause can be induced by administering Gonadotropin-releasing hormone agonists such as leuprolide acetate (or leuprorelin) and goserelin to induce ovarian suppression, by radiation therapy aimed at the ovaries or by oophorectomy (i.e. surgical removal of one or both of a woman's ovaries).

For the purpose of clarity and a concise description, features are described herein as part of the same or separate aspects and preferred embodiments thereof, however, it will be appreciated that the scope of the invention may include embodiments having combinations of all or some of the features described.

The invention will now be illustrated by the following examples, which are provided by way of illustration and not of limitation and it will be understood that many variations in the methods described and the amounts indicated can be made without departing from the spirit of the invention and the scope of the appended claims.

TABLE 1
Overview of MammaPrint probes and signature genes.

| | Probe sequence | Gene | Ensemble ID | REF SEQ ID | Corr |
|---|---|---|---|---|---|
| 1 | CTGAGTGGTCAGAGATCTGTAAAGCATGACTTTCAAG | ALDH4A1 | ENSG00000159423 | NM_170726 | + |
| | GATGGTTCTTAGGGGACTGTGTA | | | | |
| 2 | AGGACTTGAATGAGGAAACCAACACTTTGAGAAACCA | FGF18 | ENSG00000156427 | NM_003862 | + |
| | AAGTCCTTTTTCCCAAAGGTTCT | | | | |
| 3 | GCCATTAAGATTTGGATGGGAAGTTATGGGTAATGAG | CAPZB | ENSG00000077549 | NM_017765 | + |
| | AATATAATGACATCTTGCAACAT | | | | |
| 4 | GATGGCCCAGCCTGTAAGATACTGTATATGCGCTGCTG | BBC3 | ENSG00000105327 | NM_014417 | + |
| | TAGATACCGGAATGAATTTTCT | | | | |
| 5 | GGCCTCACATTCTGCTCTGCTAAGTTTGGAGAAAACAG | EBF4 | ENSG00000088881 | XM_938882 | + |
| | AACAATAAACCAGATGCAGGTG | | | | |
| 6 | AAGTACTGGAATGTAATGGTTGAAATTCCTATTCAGT | NA | NA | NT_022517 | + |
| | GATCTGGAAGAACTCTAATGTTC | | | | |
| 7 | CCAACGCACACCAGTCTTCTCAATCTGACTGTAATCTA | MYLIP | ENSG00000007944 | NM_013262 | + |
| | ATCTGTTGTGCTTTTGTTGGAC | | | | |
| 8 | GGTTTAAAGCTGAAGAGGTTGAAGCTAAAAGGAAAAG | WISP1 | ENSG00000104415 | NM_003882 | + |
| | GTTGTTGTTAATGAATATCAGGC | | | | |
| 9 | GGCTAAAAGGGAAAAAGGATATGTGGAGAATCATCAA | GSTM3 | ENSG00000134202 | NM_000849 | + |
| | GATATGAATTGAATCGCTGCGAT | | | | |
| 10 | CCTTTCAAACATGATCAAAGATTTCCCAATGTGATCTC | RAB27B, | ENSG00000041353, | NM_004163 | + |
| | ATCATCATGGATACTCAATTTG | AC098848.1 | ENSG00000267112 | | |
| 11 | GGGGAACAATGAGGGCATTTCATGAACCATCTCAGGCA | RTN4RL1 | ENSG00000185924 | NM_178568 | + |
| | CTTCTGCATCACGGAAGACCTG | | | | |
| 12 | TGCCTTGAGAATTTCAAAAGAGGTAATCAGGAAAAGA | ECI2 | ENSG00000198721 | NM_006117 | + |
| | GAGAGAGAAAAACTACACGCTGT | | | | |
| 13 | GTCTGGGATTAAGGGCAAATCTATTACTTTTGCAAACT | TGFB3 | ENSG00000119699 | NM_003239 | + |
| | GTCCTCTACATCAATTAACATC | | | | |
| 14 | TAAAAAAGAAATAGTCAGTGTTTTCCTCCTTTCAACCG | STK32B | ENSG00000152953 | NM_018401 | + |
| | AGACTATTTCTGGATTGTGTGC | | | | |
| 15 | TTTTCAGAAAGAAGTCTGGACCAGGCTGAAGGCATTT | ECI2 | ENSG00000198721 | NM_206836 | + |
| | GCAAAGCTTCCCCCAAATGTCTT | | | | |
| 16 | CCTCATTGCCTTATTCGGAGTACTATTATCCAATATAT | MS4A7, | ENSG00000166927; | NM_206939 | + |
| | GAAATCAAAGATTGTCTCCTGA | MS4A14 | ENSG00000110079 | | |
| 17 | TGGCATCATACAAAGAGCAGGAGAAGCAAACACCCAG | AP2B1 | ENSG00000006125 | NM_1030006 | + |
| | AACTCTTTTGCTGGTCAGAGATT | | | | |
| 18 | TCCAGACCTACCTTGTACGCACATAGACATTTTCATAT | DHX58 | ENSG00000108771 | NM_024119 | + |
| | GCACTGGATGGAGTTAGGGAAA | | | | |
| 19 | ATCTTTGTTAATTATTTTGGGGAGTAGTTGGGAAATG | RAI2 | ENSG00000131831 | NM_021785 | + |
| | GAAAGGTGAATTGGCTCTAGAGG | | | | |
| 20 | GTTCATTTCCAGCCCTTTCTAGATCTGATCTTTTAGGG | HIPK2 | ENSG00000064393 | NT_007933 | + |
| | GGAAAGACAGCTTAAAATGTTC | | | | |
| 21 | TGAATGTCATGTTTATGTCATAGACGTAGAAAACGCA | QDPR | ENSG00000151552 | NM_000320 | + |
| | TCCTTGAATTAAACTGCCTTAAC | | | | |
| 22 | TACTGGAGTAACTGAGTCGGGACGCTGAATCTGAATCC | ZG16B | ENSG00000162078 | NM_145252 | + |
| | ACCAATAAATAAAGCTTCTGCA | | | | |
| 23 | CAGATTCCCCAGAAACTACCTTTTGCCCAAAGAACATG | NE01 | ENSG00000067141 | NM_002499 | + |
| | CTCAGTATTTGGGGCATTTCCT | | | | |
| 24 | AGGCAGGGGTGGTGATTCATGCTGTGTGACTGACTGTG | ACADS | ENSG00000122971 | NM_000017 | + |
| | GGTAATAAACACACCTGTCCCC | | | | |
| 25 | TGGATTTCTAAACTGCTCAATTTTGACTCAAAGGTGCT | BTG2 | ENSG00000159388 | NM_006763 | + |
| | ATTTACCAAACACTCTCCCTAC | | | | |
| 26 | CCAATCCAACAACTATAGGCTGGGTTAAATAAAAGGT | BBOF1, | ENSG00000119636, | NM_005589 | + |
| | CATTATTGTCTATATTCCAAGTG | ALDH6A1 | ENSG00000119711 | | |
| 27 | TCTACCACATTAAATTCTCCATTACATCTCACTATTGG | LYPD6, | ENSG0000018712 | NM_194317 | + |
| | TAATGGCTTAAGTGTAAAGAGC | LINC00474 | ENSG00000204148 | | |
| 28 | TGAGGAATTCTTGTACGCAGTTTTCTTTGGCTTTACGA | CIRBP | ENSG00000099622 | NM_001280 | + |
| | GCCGATTAAAAGACCGTGTGAA | | | | |
| 29 | CTGGTCTTTGAAAGAAATGTACTACTAAAGAGCACTA | AC07914, | ENSG00000227210 | NM_002381 | + |
| | GTTGTGAATTTAGGGTGTTAAAC | MATN3 | ENSG00000132031 | | |
| 30 | ATGATGGGAGAGCTCTGGCAGATGTCCCAATCCTGGAG | INPP5J | ENSG00000185133 | NM_014422 | + |
| | GTCATCCATTAGGAATTAAATT | | | | |
| 31 | CAACTTGCTCTTTCATATGAGTTGGTCATAGCATGTAA | FGD6 | ENSG00000180263 | NT_019546 | + |
| | GAACCAATCTTGAAATATCGTT | | | | |
| 32 | CCTGGATCAGAGTAAGAATGTCTTAAGAAGAGGTTTG | CACNA1D, | ENSG00000157388, | NT_022517 | + |
| | TAAGGTCTTCATAACAAAGTGGT | CHDH | ENSG00000016391 | | |
| 33 | CTCCTGGACTGCTTCTTTTGGCTCTCCGACAACTCCGG | SDSL | ENSG00000139410 | NM_138432 | + |
| | CCAATAAACACTTTCTGAATTG | | | | |
| 34 | AACCAACCCATAATTGCATTTTACTTGTCGTGGTTCGA | MINOS1, | ENSG00000173436, | NM_ | + |
| | TCTGATTGTATTGTCGAAGGAC | NBL1, | ENSG00000158747, | 001032363 | |
| | | MINOS1-NBL1 | ENSG00000270136 | | |
| 35 | ATTCCTTTATGAGCTCTCCATATCCTTCTTGAGAAACT | PEX12 | ENSG00000108733 | NM_000286 | + |
| | GGTTAAAAAAGGAATAGGGGTA | | | | |
| 36 | AGTGGGGGTTGTGTAAAGGGGAAGTCATCTTTTGAGA | ERGIC1 | ENSG00000113719 | NM_ | + |
| | TCCAGATAGACATGGTTTGTGCA | | | 001031711 | |
| 37 | TCAGCTTAAGTACTTATTGTGGTAGTGAGTCCTACGGT | FBX016 | ENSG00000214050 | NM_172366 | + |
| | ATTTCAGTAAAAAGGAATTCAT | | | | |
| 38 | GGCAAGAGTTATCATAGAACAACAAAATAGAGTGGAC | ZNF385B | ENSG00000144331 | NM_152520 | + |
| | TCTTTTAGAGCATCTATATCTGC | | | | |
| 39 | GGAGTTTCTGTTTAGGGCATTAAAAATTCCCGCAAACT | IP6K2 | ENSG00000068745 | NM_1005913 | + |
| | ATAAAGAGCAATGTTTTCAGTC | | | | |
| 40 | ATAATTCTCTGTACAGGGGGGTTTGTGCTATACACTGG | MARCH8 | ENSG00000165406 | NM_145021 | + |
| | GATGTCTAATTGCAGCAATAAA | | | | |
| 41 | AGGACTTTAATCTTGGTGATGCCTTGGACAGCAGCAAC | CMTM8, | ENSG00000170293, | NM_199187 | + |
| | TCCATGCAAACCATCCAAAAGA | KRT18P15, | ENSG00000234737, | | |
| | | KRT18P34, | ENSG00000244515, | | |
| | | KRT18P13, | ENSG00000214417, | | |
| | | PCDH11Y, | ENSG00000099715, | | |
| | | KRT18P10 | ENSG00000214207 | | |
| 42 | GGGCAAAATGTATCACTCCAAACACTACTGATTCAGCA | RUNDC1 | ENSG00000198863 | NM_173079 | + |
| | TTGTTTTCATGTCTTAAAATTG | | | | |
| 43 | CTGGATGTTTAGCTTCTTACTGCAAAAACATAAGTAA | TBC1D9 | ENSG00000109436 | NM_015130 | + |
| | AACAGTCAACTTTACCATTTCCG | | | | |
| 44 | GGTAACTTGCAGGAATATTCTATTGGAAAAGATAACA | LETMD1 | ENSG00000050426 | NM_015416 | + |
| | GGAAGTACAAGTGCTTCTTGACC | | | | |
| 45 | TCAATGGTTAGCAGAAGGGAGAAAAGAAAGCAGGAAA | RILPL2 | ENSG00000150977 | NP_659495 | + |
| | ATGTGCTATTGAGATTCCAGTGG | | | | |
| 46 | CCTGGGTTTACAACGCTGTTAGGAAAATTAACCAATGA | SEC14L2, | ENSG00000100003, | NM_012429 | + |
| | ATAAAGCAACGTTCAGTGCGCA | AC004832.3 | ENSG00000249590 | | |
| 47 | TTTTTGTACCTTGTCACTATAACTACTTCCTAGTCAAA | KIAA1217 | ENSG00000120549 | NM_019590 | + |
| | GAACGAAATGTAACTGTTACCG | | | | |
| 48 | TTCTAGCTGTTATTTTGCTATTTGGCATTTACATAAAA | CCDC74A,, | ENSG00000163040, | NM_207310 | + |
| | GCACACGATGAAGCAGGTATCG | MED15P9 | ENSG00000223760, | | |
| | | CCDC74B | ENSG00000152076 | | |
| 49 | TTGGGTTTATTTCCAGGTCACAGAATTGCTGTTAACAC | TBX3 | ENSG00000135111 | NM_005996 | + |
| | TAGAAAACACACTTCCTGCACC | | | | |
| 50 | GAACAGCTCCTTACTCTGAGGAAGTTGATTCTTATTTG | FUT8 | ENSG00000033170 | NM_178157 | + |
| | ATGGTGGTATTGTGACCACTGA | | | | |
| 51 | CTTTCTTATTTACTAAGAATTTGCCTGTTTGAATAAGA | KIF3B | ENSG00000101350 | NM_004798 | + |
| | ACAAAACGCTAAGGTGGGTAGC | | | | |
| 52 | CTAGAGAGCAGAAATAAAAAGCATGACTATTTCCACC | PCAT7, FBP1 | ENSG00000231806, | NM_000507 | + |
| | ATCAAATGCTGTAGAATGCTTGG | | ENSG00000165140 | | |
| 53 | GTTCAGGGGCATCACCTACTTTGCTTACTTGATTCAAG | LBHD1, | ENSG00000162194, | NM_024099 | + |
| | GCTCTCATTAAAGACATTTTAG | CSKMT | ENSG00000214756 | | |
| 54 | GTTGGTAGAGGGAGTATGATAAAATGTTTAAATCTCA | KIAA1324 | ENSG00000116299 | NM_020775 | + |
| | TTTGGTTACCTTGAGTCCTGGAA | | | | |
| 55 | AATTCAACAGTGTGGAAGCTTTAGGGGAACATGGAGA | TMEM25 | ENSG00000149582 | NM_032780 | + |
| | AAGAAGGAGACCACATACCCCAA | | | | |
| 56 | CAAGTTGTGCAAAGTGAGAAAGATCTTTGTGGGCACA | PIN4, RPS4X | ENSG00000102309, | NM_001007 | + |
| | AAAGGAATCCCTCATCTGGTGAC | | ENSG00000198034 | | |
| 57 | CAAGAGAACCTGGAGAAAACTACCGTATTCAAGAGAT | STON2 | ENSG00000140022 | NT_026437 | + |
| | TAATCAAAATCAGTGTTTTAGCC | | | | |
| 58 | CCGAATGACCTTAAAGGTGATCGGCTTTAACGAATATG | TENM3 | ENSG00000218336 | XM_940722 | + |
| | TTTACATATGCATAGCGCTGCA | | | | |
| 59 | AGTTTATGGGCCAGAATATTCTGTATACCAGACATTGG | RASL11B | ENSG00000128045 | NM_023940 | + |
| | TAAGCTCTCATGGTTTACAGGA | | | | |
| 60 | CCATGTGGCCAGTCTACCATGGGGCCCAGGAGTTGGGG | GSDMD | ENSG00000278718 | NM_024736 | + |
| | AAACACAATAAAGGTGGCATAC | | | | |
| 61 | ATGCTTAAACCCACGGAAGGGGGAGACTCTTTCGGATT | LAMP5 | ENSG00000125869 | NM_012261 | + |
| | TGTAGGGTGAAATGGCAATTAT | | | | |
| 62 | TTCTTTCTTCAAAGAGTCATCAGAATAACATGGATTGA | CHPT1, | ENSG00000111666, | NM_153694 | + |
| | AGAGACTTCCGAACACTTGCTA | SYCP3 | ENSG00000139351 | | |
| 63 | TGAAGTCAGCGTTAACCATGTGCATACAACTTAAGGA | ZNF627 | ENSG00000198551 | NM_145295 | + |
| | ATTTTTTCCTCCTCATGTAAATT | | | | |
| 64 | GTTAAACAGGGATTATAGTACTTGTCTCACAAAGTTTC | COL23A1 | ENSG00000050767 | NT_023133 | + |
| | TGTGAGAATTAAACAAGGGGAT | | | | |
| 65 | CAGCCTGTGTGATACAAGTTTGATCCCAGGAACTTGAG | SCUBE2 | ENSG00000175356 | NM_020974 | + |
| | TTCTAAGCAGTGCTCGTGAAAA | | | | |
| 66 | AATGCACAGATCTGCTTGATCAATTCCCTTGAATAGGG | AC023024.1, | ENSG00000259172, | NM_138319 | + |
| | AAGTAACATTTGCCTTAAATTT | PCSK6 | ENSG00000140479 | | |
| 67 | TTTCCAATAACCACCTAAATTTTAACAAAGGTTCCTTC | RBP3 | ENSG00000265203 | NM_002900 | + |
| | TAAGTGGTAGAACTTGGGGTGG | | | | |
| 68 | AGTTATGCTTCCCTTCATGTTATATGCACATTGCCAAG | MYRIP, | ENSG00000170011, | NM_015460 | + |
| | AATTACTGTCAAGAGAAATGAT | EIF1B-AS1 | ENSG00000280739 | | |
| 69 | AAGGTTTGAAGGTTACGGCTCAGGGCTGCCCCATTAAA | SPEF1 | ENSG00000101222 | NM_015417 | + |
| | GTCAGTGTTGTGTTCTAAAAAA | | | | |
| 70 | GGACTGTATGAATTTATAGAAAATTGAATCTAATTTC | CLSTN2 | ENSG00000158258 | NT_005612 | + |
| | AGAAGAGCGCACTGTCTTCTCAG | | | | |
| 71 | TACATTTCTTTGGGTTTCTAGAGACGCCCCTAAGTCAC | EVL,DEGS2 | ENSG00000196405, | NM_016337 | + |
| | CTGCTTCATTAGACGGTTTCCA | | ENSG00000168350 | | |
| 72 | GGCCTAATTGAGGGAAGGAGGAAATTCATACCAGCAG | ELMOD3 | ENSG00000115459 | NM_032213 | + |
| | TTTTCAAATAAAAGAATTGTTCT | | | | |
| 73 | TCCAATTCTACACTCAGTTAAAGACCATTACTTCTCAG | BBOF1, | ENSG00000119636, | NM_005589 | + |
| | TGGAAAGAAGAAGATGCTACTC | ALDH6A1 | ENSG00000119711 | | |
| 74 | GTGGGGACTTCGTGGGAGGCACTCATGGCTCTCTGGGT | KIAA1683 | ENSG00000130518 | NM_025249 | + |
| | CTAATGAATAAAGTCCTCCACA | | | | |
| 75 | CCAGGATCTTAAGGAAGAATATTCTAGGAAGAAGGAA | SPC25 | ENSG00000152253 | NM_020675 | − |
| | ACTATTTCTACTGCTAATAAAGC | | | | |
| 76 | AGAAAACCCTTTTCTACAGTTAGGGTTGAGTTACTTCC | TFRC | ENSG00000072274 | NM_003234 | − |
| | TATCAAGCCAGTACGTGCTAAC | | | | |
| 77 | TAGGGAATGAATGAATGAATATGGATTGCTGTTAACT | PAQR3 | ENSG00000163291 | NM_177453 | − |
| | AGAAACACTTCTGTATGTCAGTC | | | | |
| 78 | GTACTTAGCTGGAAGAACATGTTAATTCTGCAATATG | MLLT10 | ENSG00000078403 | NM_1009569 | − |
| | TTTCTTGGTTAAACATTGCACAG | | | | |
| 79 | ACTCTCTTAGGTCATTTTTCAATGTGTGTAACCAAAAG | CENPBD1 | ENSG00000177946 | NM_145039 | − |
| | TTAATCAGAATAAAGCGGAAGC | | | | |
| 80 | AATGCTTTGTTGGAGTTTAAAAATTCAGGGAAAAAAT | AL44926, | ENSG00000274068, | NM_013296 | − |
| | CGGCAGACCATTAGTTACTATGG | GPSM2, | ENSG00000121957, | | |
| | | CLCC1 | ENSG00000121940 | | |
| 81 | AAGAAACCAGCATGTGACTTTCCTAGATAACACTGCTT | PIMREG | ENSG00000129195 | NM_019013 | − |
| | TCTCATAATAAAGACTATTTGC | | | | |
| 82 | GTTGGCATTGATATGGTACAACCTGCAAATTACTTGCA | HACD2 | ENSG00000206527 | NM_198402 | − |
| | GTTCTGAGTTTCAGATAAAACA | | | | |
| 83 | AGTGTCATTTTAAGGGACATTTTTATGACTTTTATGTG | ACE, | ENSG00000159640, | NM_152831 | − |
| | TATGTTTATGTAGAAATTTGGA | AC113554 | ENSG00000264813 | | |
| 84 | ACTCACTTCTTTTCAGGTGTAGCTACAATTGTGTAATG | OXCT1 | ENSG00000083720 | NM_000436 | − |
| | TACAATATTAGAGAAAGGACAG | | | | |
| 85 | CCTGGGAGCAAATGAACAATAGCTAAGTGTCTTGGTA | GNAZ | ENSG00000128266 | NM_002073 | − |
| | TTTAAAGAGTAAATTATTTGTGG | | | | |
| 86 | CCAAGAATATATGCTACAGATATAAGACAGACATGGT | FLT1 | ENSG00000102755 | NM_002019 | − |
| | TTGGTCCTATATTTCTAGTCATG | | | | |
| 87 | ATGCTTTCCTAAATCAGATGTTTTGGTCAAGTAGTTTG | MAD2L1, | ENSG00000164109, | NM_002358 | − |
| | ACTCAGTATAGGTAGGGAGATA | MNAT1 | ENSG00000020426 | | |
| 88 | ATTTGTGTGGACAAAAATATTTACACTTAGGGTTTGG | CDC25B | ENSG00000101224 | NM_004358 | − |
| | AGCTATTCAAGAGGAAATGTCAC | | | | |
| 89 | AAATATACTATGTTTGCGAACCTTGGTAGCTATGATG | KIF21A | ENSG00000139116 | NM_017641 | − |
| | AGAGCTATTATCATCTGTGGTGG | | | | |
| 90 | TCAATGAAAGTTCAAGAACCTCCTGTACTTAAACACGA | HMGB3 | ENSG00000029993 | NM_005342 | − |
| | TTCGCAACGTTCTGTTATTTTT | | | | |
| 91 | ACCTTGATAGTTCACCACGTCTGATGGATCCCTGTTTT | PTDSS1 | ENSG00000156471 | NM_014754 | − |
| | AAATAAAAACGATTCACTTTAA | | | | |
| 92 | TAAAATACTTCAATCCTGGATTCACAGTGGGAACAAG | MTMR2 | ENSG00000087053 | NM_016156 | − |
| | TTTCTATTAAAAGGCAAATGCTG | | | | |
| 93 | GGCTGTGAACAATGTTAAATAGCATCAGTTTGTCCAAT | CENPU | ENSG00000151725 | NM_024629 | − |
| | AGTTTTAAAGGCCATAATCATC | | | | |
| 94 | ACGAGTACCGGCATGTTATGTTACCCAGAGAACTTTCC | AL353705 | ENSG00000234819 | NM_001827 | − |
| | AAACAAGTACCTAAAACTCATC | | | | |
| 95 | ATTTTTTAGAAAATACACACTTTTCAGGAGAAACCTG | C1orf198 | ENSG00000119280 | NM_032800 | − |
| | AGCATGATTTTGGATTCTCCACC | | | | |
| 96 | CAGCTCAGACCATTTCCTAATCAGTTGAAAGGGAAACA | RRM2, | ENSG00000171848, | NM_001034 | − |
| | AGTATTTCAGTCTCAAAATTGA | AC007240 | ENSG00000284681 | | |
| 97 | CACTGCAGACTCTCAAGAGATCAATCAAATTGCCAGAA | INTS7 | ENSG00000143493 | NM_015434 | − |
| | ACAGTTTGGTTTTTCATATGGA | | | | |
| 98 | TGAAACTTTCTTCTGATGAGTTTCTTTAACGTACAGGA | MRPL13 | ENSG00000172172 | NM_014078 | − |
| | TGGAGTAAAACAAATGGTACAG | | | | |
| 99 | CAATTCTTGAGAGTTAATGTGATCATGATATTGCAAA | ARMC1 | ENSG00000104442 | NM_018120 | − |
| | CAACTATAAATGGTCTCTAGGCC | | | | |
| 100 | GAAGGAAACACCGAGTCTCTGTATAATCTATTTACATA | ADM | ENSG00000148926 | NM_001124 | − |
| | AAATGGGTGATATGCGAACAGC | | | | |
| 101 | AGCAACCTGGGCCTTGTACTGTCTGTGTTTTTAAAACC | IGFBP5 | ENSG00000115461 | NM_000599 | − |
| | ACTAAAGTGCAAGAATTACATT | | | | |
| 102 | GGGAATTTGATGCAGTAAAGATTACCCTGTTTTATGA | SKA3 | ENSG00000165480 | NM_145061 | − |
| | TTGTTCCTTGAAAGTCAAATGGG | | | | |
| 103 | TAAGGCTAATGATACCAATGAGGGTTGGTTTATTATC | SLC7A1 | ENSG00000139514 | NM_003045 | − |
| | AAACCTGAATAGCTGTGGTTTCT | | | | |
| 104 | TGGGGAGATACATCTTATAGAGTTAGAAATAGAATCT | PRAME | ENSG00000185686 | NM_006115 | − |
| | GAATTTCTAAAGGGAGATTCTGG | | | | |
| 105 | TATCTTGAAACTGACCAAACGCTTATTGTGTAAGATA | CTSV | ENSG00000136943 | NM_001333 | − |
| | AACCAGTTGAATCATTGAGGATC | | | | |
| 106 | TTCTCTGAAGGAATCATGTTCAGTGTTCGACCACCTAA | SMC4P1 | ENSG00000229568, | NM_1002799 | − |
| | GAAAAGTTGGAAAAAGATCTTC | AC07959 | ENSG00000248710, | | |
| | | SMC4 | ENSG00000113810, | | |
| | | TRIM59 | ENSG00000213186 | | |
| 107 | TGTCATAGACATGTATTGGGGAGCTTCCAATTAGCATA | NIPA1 | ENSG00000170113 | NM_144599 | − |
| | CATAGACACATGTGTCAGTGGC | | | | |
| 108 | TGTCCATGCTACAAGAAGTTATGAGCCTTGTTCTAAGT | SFT2D2 | ENSG00000213064 | NM_005149 | − |
| | ACAGATGAACCTTGTATTTGTG | | | | |
| 109 | ATCCCGATTTCAGTCAGACAAATACTCATTTCAGAGAT | SACS | ENSG00000151835 | NM_014363 | − |
| | TCTATACTTCATGGAATCAAGA | | | | |
| 110 | AGTTACTTTCTTAATGTGACCTAGCAATAGGCATAGCT | CTPS1 | ENSG00000171793 | NM_001905 | − |
| | ACGTGGCACTATATTCTGGCCA | | | | |
| 111 | GAAATCTCTCTACACAGATGAGTCATCCAAACCTGGGA | NUSAP1 | ENSG00000137804 | NM_018454 | − |
| | AAAAATAAAAGAACTGCAATCA | | | | |
| 112 | AAATTGCTAAGTGGAATGCATGAATTGCATTATGTTC | PSMD7 | ENSG00000103035, | NM_002811 | − |
| | TCTGGTAACACGTAGAGTTCAGA | AC009120 | ENSG00000259972 | | |
| 113 | CCAAAGGTCTTGGTACAACCAGCTGCCCATTTTGTGAA | BUD23 | ENSG00000071462, | NM_004603 | − |
| | ATTTTTATGTAGAATAAACATT | STX1A | ENSG00000106089 | | |
| 114 | GTTTCGGGTCTTTACCTCATAGTATGAAATTAGTAAGA | KIAA1147 | ENSG00000257093 | XM_1130020 | − |
| | CACTGCATAGATTTTGCCCTGA | | | | |
| 115 | GAGTACGGATGGGAAACTATTGTGCACAAGTCTTTCCA | NDRG1 | ENSG00000104419 | NM_006096 | − |
| | GAGGAGTTTCTTAATGAGATAT | | | | |
| 116 | TATTTTATCAGCACTTTATGCACGTATTATTGACATTA | PFKP | ENSG00000067057, | NM_002627 | − |
| | ATACCTAATCGGCGAGTGCCCA | AL45116 | ENSG00000278419 | | |
| 117 | TGCCCTATGGAAAACTTGTCCAAATAACATTTCTTGAA | CD163L1 | ENSG00000177675 | NM_174941 | − |
| | CAATAGGAGAACAGCTAAATTG | | | | |
| 118 | CTCCTTGTCATTGACCTTAGCTAAACCATGGCAATTCA | MAPRE2 | ENSG00000166974 | NM_014268 | − |
| | TAAATAGAGGAAACATTAATGA | | | | |
| 119 | CTGAACGAGAACAAGAATCAGAAGAAGAAATGTGACT | TMEM45A | ENSG00000181458 | NM_018004 | − |
| | TTGATGAGCTTCCAGTTTTTCTA | | | | |
| 120 | TATATTATCAGTCTGTACCAGTAGACCAGTACCCTAAC | PABPC1 | ENSG00000070756 | NM_002568 | − |
| | TACTGAAAAGAATATGGCAGTT | | | | |
| 121 | AGTAACGCTAACTTTGTACGGACGATGTCTCATGGATT | RHBDF2 | ENSG00000129667 | NM_ | − |
| | AAATAATATTCTTTATGGCAGT | | | 001005498 | |
| 122 | GTGGATCTACCTCAGTTAAACAGTTGGGTGCTATTACT | AGO2 | ENSG00000123908 | AF093097 | − |
| | AAGTCTGTCAAATTAAATTGGA | | | | |
| 123 | CATTCTAAAGGGAAATCAGTAAAATGTCTTGATAATT | TMEM64 | ENSG00000180694 | NM_1008495 | − |
| | GGTATCCAAATCACTTGTGTGCC | | | | |
| 124 | CCAAAGACAAACGATTAGAAGATGGCTATTTCAGAAT | MGAT4A | ENSG00000071073 | NM_012214 | − |
| | AGGAAAATTTGAGAATGGTGTTG | | | | |
| 125 | CAAACTTCCTGACACTACTTCCATATTTGCACTAAAGG | CDK16 | ENSG00000102225 | NM_006201 | − |
| | AGATTCAGCTACAAAAGGAGGC | | | | |
| 126 | ACCTTCCTATGAAGATCATGGAATCAAATACGGGACAT | AL589666 | ENSG00000271793, | NM_006372 | − |
| | TGAACTAATACTTGGACTTTGA | SYNCRIP | ENSG00000135316 | | |
| 127 | GGCTAACACAATCTAATTTTGGTTTAAGAGACAAATC | HIF1A | ENSG00000100644 | NM_181054 | − |
| | TAGAGTCTCAAATGATCTCAGAG | | | | |
| 128 | TGGACCCTTAAATATGACTAAAATCACAGCAATATTGT | RRAGD | ENSG00000025039 | NM_021244 | − |
| | TACATACGGGTTATATGCCAAC | | | | |
| 129 | TAAGCATTGTGAAGGAAGATTAATATAGCCAAATAAC | HIF1A | ENSG00000100644 | NM_181054 | − |
| | TAGAGTGATCAGTTCTACCAGAG | | | | |
| 130 | CCTGGATAAAAGTACTGTATGATTTTGTGATGGATGA | DEGS1 | ENSG00000143753 | NM_144780 | − |
| | TACAATAAGTCCCTACTCAAGAA | | | | |
| 131 | GCTTTGTTACTTTGTTAGGTACGAATCACATAAGGGAG | LRP12 | ENSG00000147650 | NM_013437 | − |
| | ATTGTATACAAGTTGGAGCAAT | | | | |
| 132 | TAAAAGATGAAGAAAGCTATTAGGTATATTTGTACAT | ZDHHC20 | ENSG00000180776 | NT_024524 | − |
| | GACTGCAAATGAGTCTATGCCCG | | | | |
| 133 | GTGTGTTATCTTTATATGTCAAACTGGTTGAACACTGT | PLEKHA1 | ENSG00000107679 | NM_021622 | − |
| | AATGAGAATAAACTGCACAGAG | | | | |
| 134 | GATTATTGTACGAAGTGTCTCTGTAATTATCATACTAC | FBX05 | ENSG00000112029 | NM_012177 | − |
| | TAAAGACTGTTCAGATGGCAAG | | | | |
| 135 | CATTTGTATTAATGGAATACTAAGTCCCTCTGTGATTT | NEAT1 | ENSG00000245532 | NT_033903 | − |
| | CTGAACCAAGCTATTCCTAGGC | | | | |
| 136 | ATGAAGAGATTTCTCAAGCTATTCTTGATTTCAGAAAC | PIR | ENSG00000087842 | NM_003662 | − |
| | GCAAAAAATGGGTTTGAAAGGG | | | | |
| 137 | AGCCAATCATGAGTACGTAAAGTGATTTTTGCTCTCTG | ASPM | ENSG00000066279 | NM_018136 | − |
| | TGTACAACTTTTAAAATCTGAC | | | | |
| 138 | ATCCTAGACCATATTTTCAAGTCATCTTAGCAGCTAGG | GBE1 | ENSG00000114480 | NM_000158 | − |
| | ATTCTCAAATGGAAGTGTTATA | | | | |
| 139 | AGTGATTTCATGCTAGAAAAATTGGAAACTAAAAGTG | HJURP | ENSG00000123485 | NM_018410 | − |
| | TGTAGCTAGGTTATTTCGGAGTG | | | | |
| 140 | GCTAAGCCAAGTAGTAGCAGTAAAACTTCTGATCCTCT | QSER1 | ENSG00000060749 | NM_024774 | − |
| | AGCATCAAAAACTACAACTACA | | | | |
| 141 | GGAAAGAAGTTGAAAGCATCTTGAAGAAAAACTCAGA | BNIP3 | ENSG00000176171 | NM_004052 | − |
| | TTGGATATGGGATTGGTCAAGTC | | | | |
| 142 | ACCTGGATATGTCTGTGAGGCTCCTGAAAGGAGACAA | AC087521 | ENSG00000254409, | BC052560 | − |
| | ATAAAGTCAATATATTTGCACAA | C11orf96 | ENSG00000187479, | | |
| | | AC087521 | ENSG00000244953 | | |
| 143 | GGGTATGAAAGATGAGTGTCTGTAAAAATCCTTCTTA | LINC00888 | ENSG00000240024 | NT_005612 | − |
| | GAAATGTATTTCCTCAAGACTCT | | | | |
| 144 | CAGATGGCAAGATTGAGTTTATTTCAACAATGGAAGG | GGH | ENSG00000137563 | NM_003878 | − |
| | ATATAAGTATCCAGTATATGGTG | | | | |
| 145 | GAAACTGTGTCACCCTAAAGAAGCATATAATCATAGC | TRIP13 | ENSG00000071539 | NM_004237 | − |
| | ATTAAAAATGCACACATTACTCC | | | | |
| 146 | CAAGCGTGTTTCTAGAGAACAGTTGAGAGAGAATCTC | STMN1 | ENSG00000117632 | NM_005563 | − |
| | AAGATTCTACTTGGTGGTTTGCT | | | | |
| 147 | CCGACAAGAGGAGATCATTTTAGATATTACCGAAATG | CENPN | ENSG00000166451, | NM_018455 | − |
| | AAGAAAGCTTGCAATTAGTGAAC | AC092718 | ENSG00000260213, | | |
| | | AC092718 | ENSG00000284512 | | |
| 148 | TAATAGCAAAATTTAACCCGTTACTCTTTAACCTTGTA | MY010 | ENSG00000145555 | NM_012334 | − |
| | CTGGAAATTCTAAGCAGTGCAG | | | | |
| 149 | CTTCCTACCTCTGGTGATGGTTTCCACAGGAACAACAG | TK1 | ENSG00000167900 | NM_003258 | − |
| | CATCTTTCACCAAGATGGGTGG | | | | |
| 150 | AAATCATTCGGTAAATCCAAACTGCTATGCAAAAGTT | RUNX1 | ENSG00000159216, | NM_004456 | − |
| | ATGATGGTTAACGGTGATCACAG | EZH2P1 | ENSG00000231300 | | |
| 151 | TTGGGTTTCTAGTCCTCCTTACCATCATCTCCATATGA | AURKA | ENSG00000087586 | NM_003600 | − |
| | GAGTGTGAAAATAGGAACACGT | | | | |
| 152 | GCTGGTGGAGTAGCAGATGATATTAATACTAACAAAA | DLGAP5 | ENSG00000126787 | NM_014750 | − |
| | AAGAAGGAATTTCAGATGTTGTG | | | | |
| 153 | TCACCCAGAACCAATGCGGTGTTTCTTAATGTTTGCAC | TBCE | ENSG00000285053, | NM_152490 | − |
| | AAATTTCCTTAAAAATCAACTT | B3GALNT2 | ENSG00000162885 | | |
| 154 | CAGGACTTCTCTTTAGTCAGGGCATGCTTTATTAGTGA | CENPF | ENSG00000117724 | NM_016343 | − |
| | GGAGAAAACAATTCCTTAGAAG | | | | |
| 155 | CCCTGTGCTATCGTAAGTTTGTTTTGAGCACTGCATTC | AL117350 | ENSG00000237481, | NM_145257 | − |
| | ACTTTAAAATTCTGGAGGAACA | CCSAP | ENSG00000154429 | | |
| 156 | CAACATATTTCAGTTGGAAAATTTGTATGCAGTAATC | ATAD2 | ENSG00000156802 | NM_014109 | − |
| | AGCCAATGTATTTATCGGCATCG | | | | |
| 157 | CCCCCATTCTGGAAGGTTTTGTTATCTTCGGAAGAACC | PSMD2 | ENSG00000175166, | NM_002808 | − |
| | CCAATTATGATCTCTAAGTGAC | FMN2 | ENSG00000155816, | | |
| | | AL359918 | ENSG00000228818 | | |
| 158 | TGTCCCCAGGGATCAAACAGAAGCAGCCGTGGGCAAAA | SHMT2 | ENSG00000182199, | NM_020142 | − |
| | TACAATTTCATTTAACAAATTG | NDUFA4L2 | ENSG00000185633 | | |
| 159 | AAACAGCATTATGGAGTTAAAAGATTTTTACAACTGG | PIMREG | ENSG00000129195, | NM_019013 | − |
| | GTCTTGATTTTGATGTGAGCTGG | PITPNM3 | ENSG00000091622 | | |
| 160 | TCCAGACGCACTGATCTTTGCAAAGGAGACTTAATTTC | DCK | ENSG00000156136 | NM_000788 | − |
| | AAATCTGTAATTACCATACATA | | | | |
| 161 | CATTTGGCTGTCAGAAATTATACCGAGTCTACTGGGTA | DTL | ENSG00000143476 | NM_016448 | − |
| | TAACATGTCTCACTTGGAAAGC | | | | |
| 162 | TTAAAGGCAAAACTGTGCTCTTTATTTTAAAAAACAC | COL4A2 | ENSG00000134871 | NM_001846 | − |
| | TGATAATCACACTGCGGTAGGTC | | | | |
| 163 | AAGGTGCTGTCATATATCTTGGAATGAATGACCTAAA | AGFG1 | ENSG00000173744 | NM_004504 | − |
| | ATCATTTTAACCATTGCTACTGG | | | | |
| 164 | GGATGTAAATCCTGAGCTCAAATCTCTGTTACTCCATT | NMB | ENSG00000197696 | NM_205858 | − |
| | ACTGTGATTTCTGGCTGGGTCA | | | | |
| 165 | CCTCAAGAGTATGTATAATTTGAAGAGATACTTTGTA | KIF14 | ENSG00000118193 | NM_014875 | − |
| | ACTATGCTTGGGTGATATTGAGC | | | | |
| 166 | TTCACAGAATAGCACAAACTACAATTAAAACTAAGCA | BIRC5 | ENSG00000089685 | NM_ | − |
| | CAAAGCCATTCTAAGTCATTGGG | | | 001012271 | |
| 167 | CCAGCACATAGGAGAGATGAGCTTCCTACAGCACAACA | VEGFA | ENSG00000112715 | NM_003376 | − |
| | AATGTGAATGCAGACCAAAGAA | | | | |
| 168 | GAGAAACATTGTATATTTTGCAAAAACAAGATGTTTG | ECT2 | ENSG00000114346 | NM_018098 | − |
| | TAGCTGTTTCAGAGAGAGTACGG | | | | |
| 169 | TACTTTTTGGAAAAGAATAAACCAAGAATTGATTGGG | IVNS1ABP | ENSG00000116679 | NM_016389 | − |
| | CACATCATTTCAAGAAGTCCCTC | | | | |
| 170 | ATGGAGTTGCTAGTAAAGCGAAGCTGATTATCCTGGA | MCCC1 | ENSG00000078070 | NM_020166 | − |
| | AAACACTATTTACCTATTTTCCA | | | | |
| 171 | GACTGCTAGTGGATAATAACATCTTGACTACTTAAAA | TMEM38B | ENSG00000095209, | NM_017779 | − |
| | AAGGGACATATTGAAAATCCTGG | AL592437 | ENSG00000232486, | | |
| | | OTUD7A | ENSG00000169918, | | |
| | | AC026951 | ENSG00000259358, | | |
| | | DEPDC1 | ENSG00000024526, | | |
| | | AL138789 | ENSG00000233589 | | |
| 172 | CATGTTACCTGGACTGGAACAGACTGTGAATATAGCAG | INAVA | ENSG00000163362, | NM_018265 | − |
| | AAGGTTCCAAGAACTCTGGTGT | SLC9C1 | ENSG00000172139 | | |
| 173 | GAGACCAGGTGCTTCAAAACTTAGGCTCGGTAGAATCT | KIF21A | ENSG00000139116 | NM_017641 | − |
| | TACTCAGAAGAAAAAGCAAAAA | | | | |
| 174 | GGATTCAACCCAAATGATTTCTCATCAGGTGATTCTTG | C16orf95 | ENSG00000260456 | AK026130 | − |
| | GTTGTAGCAAAGTTCATGTGAA | | | | |
| 175 | AGAACTCTTGATTTTGTACATAGTCCTCTGGTCTATCT | CCNB2 | ENSG00000157456, | NM_004701 | − |
| | CATGAAACCTCTTCTCAGACCA | AC092757 | ENSG00000259732 | | |
| 176 | AATTGGTAAACATCATGTTCCTGATGATAACCCAGTAG | STK3 | ENSG00000104375 | NM_006281 | − |
| | CAAAAACATTTGTACTGAGTGG | | | | |
| 177 | CATCAGTCTTGGGAAATTTGAACTTTGATCAACTTAAC | ZNF367 | ENSG00000165244 | NM_153695 | − |
| | TAAAGAAGGAAGGGTAGTAAGA | | | | |
| 178 | TTAGGGCCCTACGTAATAGGCTAATTGTACTGCTCTTA | BUB1 | ENSG00000169679 | NM_004336 | − |
| | GAATGTAAGCGTTCACGAAAAT | | | | |
| 179 | GAGTCTTTGGGATACCATTAAAAAGAAGAAAATTTCA | ASPM | ENSG00000066279 | NM_018136 | − |
| | GCCTCTACAAGTCACAACAGAAG | | | | |
| 180 | AGAGTGTGAAAAATAGGAACACGTGCTCTACCTCCATT | AURKA | ENSG00000087586 | NM_003600 | − |
| | TAGGGATTTGCTTGGGATACAG | | | | |
| 181 | AACTTTTTAGGGCAAAGTTAACACTGAAAGTTCTAGC | UTP23 | ENSG00000147679, | NM_006265 | − |
| | TTAAGTGTTGAAACTTTTGTGGG | RAD21 | ENSG00000164754 | | |
| 182 | ACTTAGCATTTTCTGCATCTCCACTTGGCATTAGCTAA | PGK1 | ENSG00000102144, | NM_000291 | − |
| | AACCTTCCATGTCAAGATTCAG | OPHN1 | ENSG0000079482, | | |
| | | AC010422 | ENSG00000269693 | | |
| 183 | TTTTGATGAGAATGAATCTTGGTACTTAGATGACAAC | CP | ENSG00000047457, | NM_000096 | − |
| | ATCAAAACATACTCTGATCACCC | LRRC69 | ENSG00000214954, | | |
| | | AC104966 | ENSG00000253525 | | |
| 184 | TTCCCTTCAATACTCCTAAAACCAAAGAAGGATATTAC | AC079781 | ENSG00000284707, | NM_183356 | − |
| | TACCGTCAAGTCTTTGAACGCC | ASNS | ENSG00000070669 | | |
| 185 | TCCTGTCCTGCTCATTATGCCACTTCCTTTTAACTGCC | CA9 | ENSG00000107159 | NM_001216 | − |
| | AAGAAATTTTTTAAAATAAATA | | | | |
| 186 | CAAAAACTCAGATCTATCTTAAGAGTGACCAGGAAGA | AL451164 | ENSG00000278419, | NM_014889 | − |
| | GGTTCATTGAAATAATCATGCAT | PITRM1 | ENSG00000107959 | | |
| 187 | CATACGGTTTTGTTTGGAGGATGGCTTCTGCTGCTAAA | TMEM74B | ENSG00000125895 | NM_018354 | − |
| | AATACAAAAGTTTGGAAACCGC | | | | |
| 188 | CAGAGGGACCTTATTTAAACATAAGTGCTGTGACTTCG | ESM1 | ENSG00000164283 | NM_007036 | − |
| | GTGAATTTTCAATTTAAGGTAT | | | | |
| 189 | GTTTGTGAAACTGTTAAGGTCCTTTCTAAATTCCTCCA | CCNE2 | ENSG00000175305 | NM_057749 | − |
| | TTGTGAGATAAGGACAGTGTCA | | | | |
| 190 | TTAACCAGCTGTAAAACACAGACCTTTATCAAGAGTAG | EGLN1 | ENSG00000135766 | NM_022051 | − |
| | GCAAAGATTTTCAGGATTCATA | | | | |
| 191 | GGGGATGAATAGAAAACCTGTAAGCTTTGATGTTCTG | CENPA | ENSG00000115163 | NM_001809 | − |
| | GTTACTTCTAGTAAATTCCTGTC | | | | |
| 192 | GTGATAAAGTACCTGATCCAAATGTTATGAGAATACT | LIN9 | ENSG00000183814 | NT_004559 | − |
| | GGACGAGAATTGAACGAAATTGA | | | | |
| 193 | TGCAGCAGTACTACTGTCAACATAGTGTAAATGGTTCT | PRC1 | ENSG00000198901 | NM_199413 | − |
| | CAAAAGCTTACCAGTGTGGACT | | | | |
| 194 | GCATGAGTCACAATTACAAAGTTTTGAGCGGTTTTGT | PALM2-AKAP2 | ENSG00000157654; | NM_147150 | − |
| | AATTTGACATTTAGGAAAGTCTC | AKAP2 | ENSG00000241978 | | |
| 195 | TTATTCGAAGACACAGAAGTTGGGCAAGTCAAATGTT | NMU | ENSG00000109255 | NM_006681 | − |
| | GTGTCGTCAGTTGTGCATCCGTT | | | | |
| 196 | TGTACTGGCAGGCTCGTTTTACCTGATTCTAGAATATT | PITRM1 | ENSG00000107959 | NM_014889 | − |
| | TAAGAATCTAAAAATAAAGGGC | | | | |
| 197 | GTGGCCTATAACTTACTTGTCAACAACTGTGAACATTT | HRASLS | ENSG00000127252 | NM_020386 | − |
| | TGTGACATTGCTTCGCTATGGA | | | | |
| 198 | CCAGGACGCCACTCATTTCATCTCATTTAAGGGAAAAA | IGFBP5 | ENSG00000115461 | NM_000599 | − |
| | TATATATCTATCTATTTGAGGA | | | | |
| 199 | CGGAGCGCAGGGTACTTGGCGTATAATAAGCCATCAAT | JHDM1D-AS1 | ENSG00000260231 | NT_007914 | − |
| | AATTTATGGGTGAAATTGAGAG | | | | |
| 200 | CAGAGCTACAACTAGGAAAATTAGAGTGGTAGTAGTC | MSANTD3 | ENSG00000066697 | NM_080655 | − |
| | ACTTATTTAAGAATTCATTCAGG | | | | |
| 201 | TTGGTAGTTAACCCTAACTACTTGCTCGAAGATTGAGA | MCM6 | ENSG00000076003 | NM_005915 | − |
| | TAGTGAAAGTAACTGACCAGAG | | | | |
| 202 | GCGTGAGCATGTCAGTATTCTAGTCCAGTATTTGCCAG | SMIM5 | ENSG00000204323 | XM_946181 | − |
| | TTTCCAAGTAAAAGCTTTTGTG | | | | |
| 203 | GCTGTGCCATTCAATGTTTGATGCATAATTGGACCTTG | CDCA7 | ENSG00000144354 | NM_031942 | − |
| | AATCGATAAGTGTAAATACAGC | | | | |
| 204 | CCAAGAAGGAAAATGTCAAAATTAGTGATGAGGGAAT | RFC4 | ENSG00000163918 | NM_181573 | − |
| | AGCTTATCTTGTTAAAGTGTCAG | | | | |
| 205 | TGCTTTAAGTGAATGGCAGTCCCTTGTCTTATTCAGAA | ORC6 | ENSG00000091651 | NM_014321 | − |
| | TATAAAATTCAGTCTGAATGGC | | | | |
| 206 | AGGTTGGCAGTAAGGCAGGGTCCCATTTCTCACTGAGA | SLC2A3 | ENSG00000059804 | NM_006931 | − |
| | AGATTGTGAATATTTCCATATG | | | | |
| 207 | GTGCAAATAGAATTAGCAGTAAGAAGCTACTCTAGCT | ADGRG6 | ENSG00000112414 | NM_1032395 | − |
| | AATTTGCCATTTCACTTAAATGG | | | | |
| 208 | GATACAGCCTACATAAAGACTGTTATGATCGCTTTGAT | MELK | ENSG00000165304 | NM_014791 | − |
| | TTTAAAGTTCATTGGAACTACC | | | | |
| 209 | CAACATTTACATTGTAATTCAATAGACGCTACTACTAC | GRHL2 | ENSG00000083307 | NT_008046 | − |
| | AAAGGAGCTTTATTCTTCCAGC | | | | |
| 210 | CAACAGTATTGCGTTGTCAGACTAGGAAAGCTAAACG | MTDH | ENSG00000147649 | NM_178812 | − |
| | AACAAAATGGTTTTAGTTTTGCT | | | | |
| 211 | CTGGTTGTCCAACTACCATATGAAGCTAGAAAATGCAC | UCHL5 | ENSG00000116750 | NM_015984 | − |
| | AAACGATATTCCTTATCTGTAA | | | | |
| 212 | GGCATCAGGGATCACATCACTCTTAACGGCTGTTACTT | RAB6B | ENSG00000154917 | NM_016577 | − |
| | AAACAACTATTTTTTGGTTTGG | | | | |
| 213 | TGAAAATGTATTTGTAGTCACGGACTTTCAGGATTCTG | ECT2 | ENSG00000114346 | NM_018098 | − |
| | TCTTTAATGACCTCTACAAGGC | | | | |
| 214 | AGACCAGGTCTCTATTTTGAGGAAGAAATACCGAGAC | EXT1 | ENSG00000182197 | NM_000127 | − |
| | ATTGAGCGACTTTGAGGAATCCG | | | | |
| 215 | AAGTCATGACACAGTATTCGCTCTTTTTCTGAATGTTT | GPR180 | ENSG00000152749 | NM_180989 | − |
| | ACATAGAGATTCATCACTGCAG | | | | |
| 216 | CAGTAAGTACGGGAAAAAATGTTTACTAACTTCCTCA | LPCAT1 | ENSG00000153395 | NM_024830 | − |
| | GAGATTCGTGATACGCGTTTCTC | | | | |
| 217 | CTTTGAATGGACATAAAAATTCTGCTTGTTAAGAACA | SERF1A | ENSG00000172058 | NM_021967 | − |
| | AGTTGAGCTCTGGTAACTGATCT | | | | |
| 218 | TGACTGATGTGTCTGAAAATGCTAAGGATCTTATTCG | CDC42BPA | ENSG00000143776 | NM_003607 | − |
| | AAGGCTCATTTGTAGCAGAGAAC | | | | |
| 219 | CTCTGAAAGAAGAAGTTCAAAAGCTGGATGATCTTTA | NDC80 | ENSG00000080986 | NM_006101 | − |
| | CCAACAAAAAATTAAGGAAGCAG | | | | |
| 220 | ATCTGTGGTTATTCGAACCTTTATTACTAGTGACTTCA | GMPS | ENSG00000163655 | NM_003875 | − |
| | TGACTGGTATACCTGCAACACC | | | | |
| 221 | TCCACCCCAGGACGCCACTCATTTCATCTCATTTAAGG | IGFBP5 | ENSG00000115461 | NM_000599 | − |
| | GAAAAATATATATCTATCTATT | | | | |
| 222 | GGCCCTCTCTTCTCACCTTTGTTTTTTGTTGGAGTGTT | MMP9 | ENSG00000100985 | NM_004994 | − |
| | TCTAATAAACTTGGATTCTCTA | | | | |
| 223 | CTGGGTTGATACCTGAAAGAATCCTGTCTTATTTGGTC | CMC2 | ENSG00000103121 | NM_020188 | − |
| | TCCATAATCCTTTGAATGGAAA | | | | |
| 224 | AGTACCCTGATATACTGAATTTTGTGGATGATTTGGA | DIAPH3 | ENSG00000139734 | NM_030932 | − |
| | ACCTTTAGACAAAGCTAGTAAAG | | | | |
| 225 | AAGACTTTCTTACTGACCTGAATAACTTCAGAACCACA | DIAPH3 | ENSG00000139734 | NM_030932 | − |
| | TTCATGCAAGCAATAAAGGAGA | | | | |
| 226 | TTTAGTGGTCCGTTGCCTCTGAAGATGTAAACAAACAA | QSOX2 | ENSG00000165661 | NM_181701 | − |
| | ATACACTATTTCTGGGAACATT | | | | |
| 227 | ATAGAATATGTATATGTATTCTTTGTCTACCAACTACC | TMEM65 | ENSG00000164983 | NT_008046 | − |
| | AAAGAAACAAATACTCCTCAGT | | | | |
| 228 | ACATTGCTTACTTAAAAGCTACATAGCCCTATCGAAAT | NUSAP1 | ENSG00000137804 | NM_018454 | − |
| | GCGAGGATTAATGCTTTAATGC | | | | |
| 229 | ACCATAAGGCAATTGAGCACATAACGAAAAATGATGC | DIAPH3 | ENSG00000139734 | NM_ | − |
| | AATAAGAATGTATGCACTCTCTT | | | 001042517 | |
| 230 | CAGCCTTTCCTCATGTCAACACAGTTCACAATATAGTT | MIR210HG | ENSG00000247095 | NT_035113 | − |
| | TTCAAAGTACAGTTTAAAACTC | | | | |
| 231 | CCTCCCCAAAATAATTAGTAACTGGTTGTTCTACTTGG | TSPYL5 | ENSG00000180543 | NM_033512 | − |
| | TAATTTGACACCCTGTTAATAA | | | | |

TABLE 2
Preferred genes.

| | Gene Name | Description | | Gene Name | Description |
|---|---|---|---|---|---|
| 194 | PALM2-AKAP2; | ENSG00000157654; | 161 | DTL | ENSG00000143476 |
| | AKAP2 | ENSG00000241978 | | | |
| 1 | ALDH4A1 | ENSG00000159423 | 5 | EBF4 | ENSG00000088881 |
| 17 | AP2B1 | ENSG00000006125 | 12 | ECI2 | ENSG00000198721 |
| 4 | BBC3 | ENSG00000105327 | 15 | ECI2 | ENSG00000198721 |
| 174 | C16orf95 | ENSG00000260456 | 213 | ECT2 | ENSG00000114346 |
| 3 | CAPZB | ENSG00000077549 | 190 | EGLN1 | ENSG00000135766 |
| 189 | CCNE2 | ENSG00000175305 | 188 | ESM1 | ENSG00000164283 |
| 218 | CDC42BPA | ENSG00000143776 | 214 | EXT1 | ENSG00000182197 |
| 203 | CDCA7 | ENSG00000144354 | 2 | FGF18 | ENSG00000156427 |
| 191 | CENPA | ENSG00000115163 | 86 | FLT1 | ENSG00000102755 |
| 223 | CMC2 | ENSG00000103121 | 220 | GMPS | ENSG00000163655 |
| 162 | COL4A2 | ENSG00000134871 | 85 | GNAZ | ENSG00000128266 |
| 160 | DCK | ENSG00000156136 | 207 | ADGRG6 | ENSG00000112414 |
| 18 | DHX58 | ENSG00000108771 | 215 | GPR180 | ENSG00000152749 |
| 229 | DIAPH3 | ENSG00000139734 | 209 | GRHL2 | ENSG00000083307 |
| 224 | DIAPH3 | ENSG00000139734 | 9 | GSTM3 | ENSG00000134202 |
| 225 | DIAPH3 | ENSG00000139734 | 217 | SERF1A | ENSG00000172058 |
| 197 | HRASLS | ENSG00000127252 | 196 | PITRM1 | ENSG00000107959 |
| 198 | IGFBP5 | ENSG00000115461 | 193 | PRC1 | ENSG00000198901 |
| 221 | IGFBP5 | ENSG00000115461 | 226 | QSOX2 | ENSG00000165661 |
| 199 | JHDM1D-AS1 | ENSG00000260231 | 212 | RAB6B | ENSG00000154917 |
| 192 | LIN9 | ENSG00000183814 | 204 | RFC4 | ENSG00000163918 |
| 216 | LPCAT1 | ENSG00000153395 | 11 | RTN4RL1 | ENSG00000185924 |
| 201 | MCM6 | ENSG00000076003 | 42 | RUNDC1 | ENSG00000198863 |
| 208 | MELK | ENSG00000165304 | 65 | SCUBE2 | ENSG00000175356 |
| 230 | MIR210HG | ENSG00000247095 | 206 | SLC2A3 | ENSG00000059804 |
| 222 | MMP9 | ENSG00000100985 | 202 | SMIM5 | ENSG00000204323 |
| 16 | MS4A7; | ENSG00000166927; | 14 | STK32B | ENSG00000152953 |
| | MS4A14 | ENSG00000110079 | | | |
| 200 | MSANTD3 | ENSG00000066697 | 13 | TGFB3 | ENSG00000119699 |
| 210 | MTDH | ENSG00000147649 | 227 | TMEM65 | ENSG00000164983 |
| 219 | NDC80 | ENSG00000080986 | 187 | TMEM74B | ENSG00000125895 |
| 195 | NMU | ENSG00000109255 | 231 | TSPYL5 | ENSG00000180543 |
| 228 | NUSAP1 | ENSG00000137804 | 211 | UCHL5 | ENSG00000116750 |
| 205 | ORC6 | ENSG00000091651 | 8 | WISP1 | ENSG00000104415 |
| 84 | OXCT1 | ENSG00000083720 | 38 | ZNF385B | ENSG00000144331 |

### 5 EXAMPLES

### Example 1

### Materials and Methods

### Samples

NSABP B42 (Mamounas et al., 2019. Lancet Oncol 20: 88-99) refers to a randomized, double-blinded, placebo-controlled clinical trial of extended adjuvant endocrine therapy (tx) with letrozole (L) in postmenopausal women with hormone-receptor (+) breast cancer (BC) who have completed previous adjuvant tx with an aromatase inhibitor (AI). A pre-treatment gene expression biomarker such as MammaPrint may identify breast cancers at risk of recurrence in years 5 to 10 after diagnosis, for whom EET will be beneficial. Tissue-based gene expression may provide clinically useful information supplemental to, or superior to, clinical and pathological features.

Total RNA was isolated from samples from the NASBP B42 trial. Samples were provided as FFPE blocks or slides. RNA extraction was performed using five sections of 5-μm thickness. The sections contained a minimum of 30% invasive carcinoma and together had an area of at least 100 mm² (50 mm2 for 10 μm slides). If necessary and possible, a manual dissection was performed to enrich for invasive cells. Discretion regarding the percentage of invasive disease down to a minimum of ˜10% can be used by the pathologist to maximize the available samples. The inclusion of Ductal Carcinoma In Situ in the sample is not known to impact the performance of the assay, as long as the minimum threshold for invasive disease was observed.

De-paraffination and total RNA extraction, including DNase treatment, was performed using the RNeasy FFPE kit (Qiagen Inc, Valencia, CA,) according to manufacturer's instructions. RNA was checked for quality and purity using the NanoDrop (Thermo Scientific, Wilmington, DE) by recording the ratio (260/280 nm). Extracted RNA was amplified using the Transplex C-WTA kit (Rubicon Genomics, Ann Arbor, MI) according to manufacturer's protocol to generate sufficient cDNA yield for microarray hybridization.

Generated cDNA was labeled with Cy3 fluorophores using the Genomic DNA Enzymatic Labeling Kit (Agilent Technologies Inc, Santa Clara CA) and hybridized to Agendia's diagnostic arrays (Agilent Technologies), both according to manufacturer's instructions. The diagnostic arrays contained all probes necessary for diagnostic readout of MammaPrint, as described previously (Glas et al., 2006. BMC Genomics 7: 278). Microarray slides were washed, scanned and gene expression levels were quantified using Feature Extraction software (Agilent Technologies). Downstream data normalization and processing including quality control was performed automatically using Agendia's proprietary software (XPrint) for readout of MammaPrint.

The MammaPrint test uses the expression levels of 70 genes to assess a patient's risk of recurrence. For the primary analysis, patient tumor samples were classified into a High Risk or Low Risk group by the 0.00 threshold in the MammaPrint index (high up to and low above 0.00 index, respectively). Recently, a subgroup of ultralow tumors is defined by MammaPrint index>+0.355 (Esserman et al., 2017. JAMA Oncol 3: 1503-1510).

All MammaPrint analyses were repeated twice. Patients included in the translational cohort were compared to other excluded B-42 patients in terms of patient and tumor characteristics, and treatment effects.

### Statistical Methods

Population for all analyses was based on the intention-to-treat (ITT) principle: patients were analyzed according to their randomized treatment, regardless of what they actually receive. This ensures the strengths of randomization are kept (an equal balance of potential prognostic factors between both groups), but it estimates the treatment effect in clinical practice.

Categorical variables are expressed in numbers and percentages. Continuous variables are expressed with median, minimum and maximum. Corresponding p-values come from Chi-squared test, Fisher's exact test or a non-parametric test to test for differences in distribution between risk groups.

Within the total study population and within each treatment arm, univariate Cox Regression Analysis were performed for MammaPrint risk groups and all available clinical, pathological, therapeutic variables. Summary statistics from the Cox model included the number of patients analyzed, hazard ratio (with 95% confidence interval (CI)) of the comparison and the according p-value. A p-value less than 0.05 (two sided) was considered statistically significance.

Differences in primary and secondary endpoints between placebo and letrozole groups were assessed by stratified log-rank tests. Stratification factors included original stratification factors from the parent B-42 trial, i.e., pathological node status at diagnosis (negative vs positive), prior tamoxifen use (no vs yes), and lowest bone mineral density T score in the lumbosacral spine, total hip, or femoral neck (≤−2.0 or >−2.0 SD).

Multivariate Cox Regression Analysis were utilized to examine the interaction between EET treatment and MP Risk index. Secondary elaborated multivariate analyses were performed to examine interaction with MammaPrint, EET treatment and any clinical and pathologic variable. Summary statistics from these Cox models include the number of patients analyzed, hazard ratios (with 95% CI) of the comparisons and the according p-values.

Comparison of the prognostic performance of the backward-selected model (p=0.05), the full model and the model without any covariates will be based upon Likelihood-Ratio index.

### Descriptive Clinical Statistics

The following clinical and pathological variables were used for descriptive summaries and appropriate analyses:
Randomized treatment arm: Letrozole vs. Placebo
Treatment before inclusion in B42: AI vs. Tamoxifen+AI
Age: continuous years
Tumor size: continuous mm
T-stage: pT1 (reference group) vs. pT2 vs. pT3
N stage: NO (reference) vs. N+
Stage: I vs. II vs. III
Tumor grading: G1 vs. G2 vs. G3
Estrogen Receptor (ER): positive vs. negative
Progesterone Receptor (PgR): positive vs. negative
Her2: positive vs. negative

### Descriptive Event and Time Statistics

The following event variables were used for appropriate analyses:
Distant Recurrence (DR): yes vs. no
Time to DR: continuous months
Overall Survival (OS): yes vs. no
Time to Death: continuous months
Disease Free Survival (DFS): yes vs. no
Time to DFS: continuous months
Breast Cancer Free Interval (BCFI): yes vs. no
Time to BCFI: continuous months

### Survival Analysis

A Kaplan-Meier curve with corresponding Log-rank test was used to visualize the survival distribution function for each MammaPrint risk group. Cox PH models stratified by nodal status, prior tamoxifen use, and lowest bone mineral density T-score was used to explore the association among risk factors listed to survival outcomes. This was done for each treatment arm. Kaplan-Meier curves were also used to visualize the survival distribution function for each treatment group.

### Results

ER+ early stage breast cancer continues to recur in years 5 to 10. Clinical trials with extended endocrine therapy show a trend to improve survival but so far have not shown a significant benefit.

NSABP-B42 assessed the effect of 5 years of letrozole after 5 years of prior endocrine therapy in postmenopausal women with early stage hormone receptor positive (HR+) breast cancer. Randomization occurred five years after diagnosis.

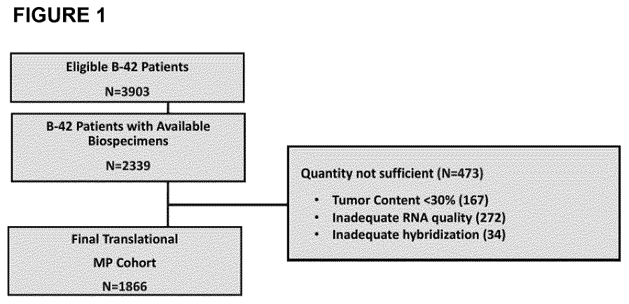

There were 3,966 patients randomly assigned to placebo or letrozole. Among them, 63 were excluded due to either no clinical assessment or not being at risk for the primary DFS endpoint for the B-42 parent trial. Blocks were available for 2,339 patients who consented for future research. The final translational MammaPrint (MP) cohort consisted of 1,866 patients with available clinical and assay data (FIG. 1). There were no differences in the distributions of patient and tumor characteristics between the translational MP cohort and the excluded B-42 cohort, except for the small differences in the HER2 status (Table 3). Compared to the excluded B-42 population, the MP cohort had slightly better prognosis in terms of DR (p=0.036) with a more pronounced letrozole effect on the rate of DR (MP cohort: HR=0.50, 95% CI 0.33-0.75; Excluded cohort: HR=0.92, 95% CI 0.66-1.29; treatment-by-inclusion status interaction p=0.03). Among 1,866 patients, 706 (37.8%) were defined as MP-H and 1,160 (62.2%) as MP-L (MP-UL: 252 [13.5%] and MP-LNUL 908 [48.7%]).

The primary endpoint for the analysis of benefit of extended endocrine therapy (EET), disease free survival (DFS indicated a beneficial effect of EET, however, this effect was hardly significant in the results obtained at 6.9 years of median follow-up (HR=0.85, 95% CI 0.73-0.999, p=0.048). In the updated results with 10.3 years of median follow-up, the beneficial effect of letrozole on DFS persisted (HR=0.85, 95% CI 0.74-0.96, p=0.01). No clinical or pathologic features identified a subset likely to benefit. DFS includes events other than distant metastatic recurrence, e.g. second breast cancers and local recurrences, and potential chemoprevention effects of EET cannot be distinguished from the reduction of distant metastases. The secondary endpoint of distant recurrence (DR) showed a 28% reduction in the letrozole arm, although no subset was identified by multivariate analysis.

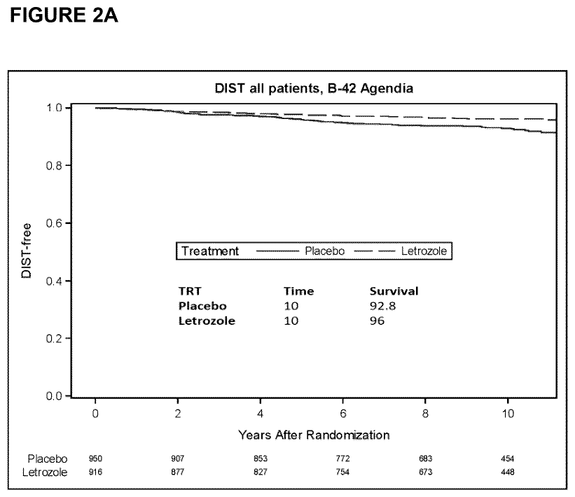

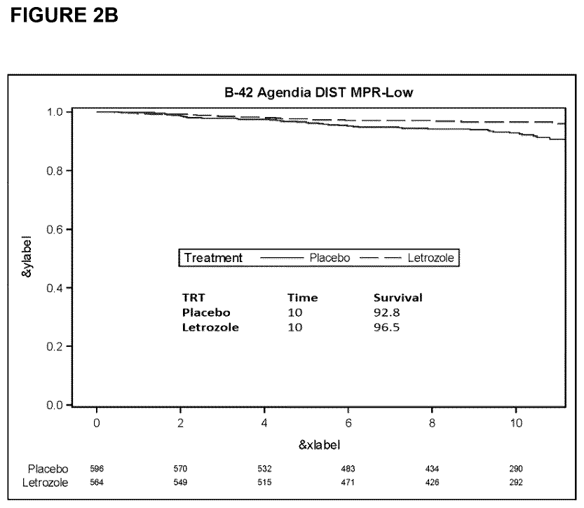

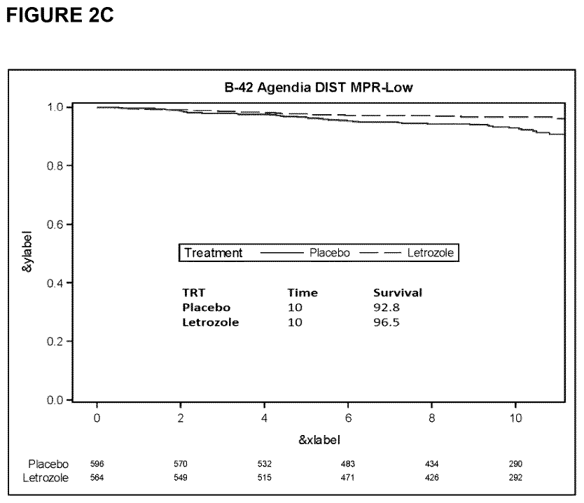

As is shown in FIG. 2, distant recurrence (FIG. 2) was significantly improved in all patients (HR=0.50 (0.33, 0.75), P=0.0007) and MammaPrint Low risk patients (HR=0.43 (0.25, 0.74), P=0.0018), but not in MammaPrint High risk patients (HR=0.65 (0.34, 1.24), P=0.19) (Table 4). MammaPrint ultralow risk patients, with a MammaPrint Index>0.355, did not show a statistically relevant improvement for Distant recurrence (HR=0.53 (0.13, 2.15), P=0.36), but Low risk patients without ultralow risk patients (with a MammaPrint Index (MPI) 0<MPI>0.355) did show a statistically relevant benefit (HR=0.42 (0.23, 0.76), P=0.003) (Table 5). The treatment-by-risk group interaction was not relevant (p=0.38).

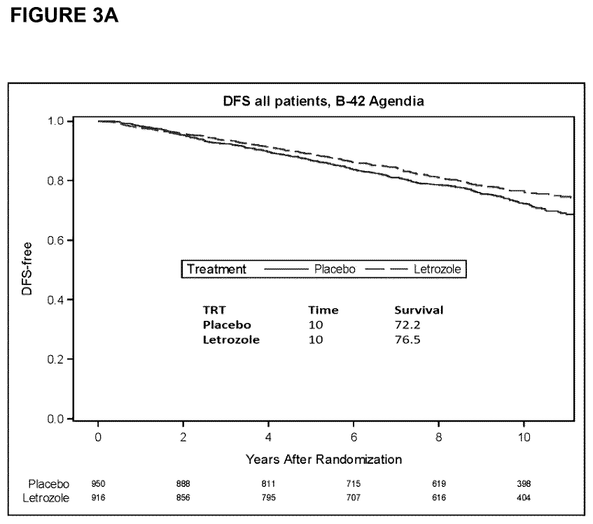

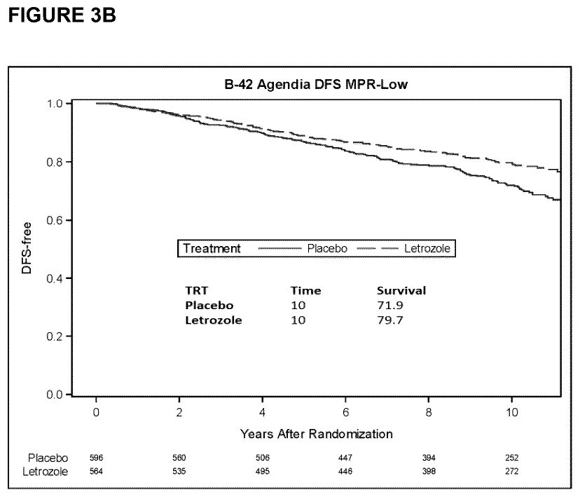

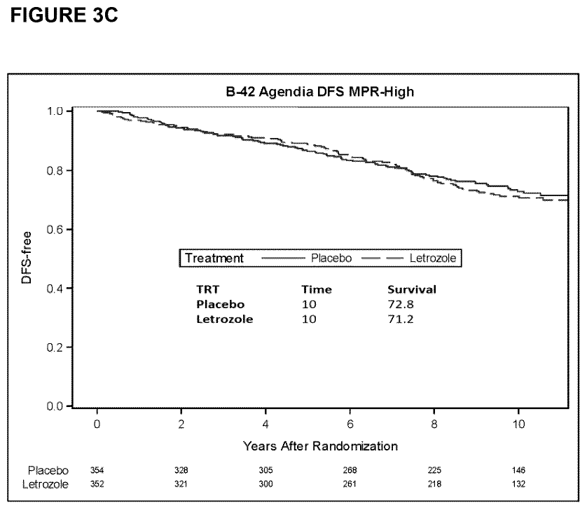

Similarly, as is shown in FIG. 3, Disease-free survival was significantly improved in all patients (HR=0.81 (0.68, 0.98), P=0.027) and MammaPrint Low risk patients (HR=0.67 (0.52, 0.85), P=0.0009), but not in MammaPrint High risk patients (HR=1.10 (0.82, 1.47), P=0.55) (Table 4). MammaPrint ultralow risk patients, with a MammaPrint Index>0.355, did not show a statistically relevant improvement for disease-free survival (HR=0.82 (0.45, 1.48), P=0.50), but Low risk patients without ultralow risk patients (with a MammaPrint Index (MPI) 0<MPI>0.355) did show a statistically relevant benefit (HR=0.64 (0.49, 0.83), P<0.001) (Table 5). The treatment-by-risk group showed a relevant interaction p=0.013.

Similarly, the breast cancer-free interval was significantly improved in all patients (HR=0.70 (0.53, 0.93), P=0.012) and MammaPrint Low risk patients (HR=0.51 (0.35, 0.74), P=0.0003), but not in MammaPrint High risk patients (HR=1.15 (0.74, 1.79), P=0.53) (Table 4). MammaPrint ultralow risk patients, with a MammaPrint Index>0.355, did not show a statistically relevant improvement for breast cancer-free interval (HR=0.67 (0.28, 1.65), P=0.38), but Low risk patients without ultralow risk patients (with a MammaPrint Index (MPI) 0<MPI>0.355) did show a statistically relevant benefit (HR=0.48 (0.32-0.73), P=0.003) (Table 5). The treatment-by-risk group showed a relevant interaction with p=0.006.

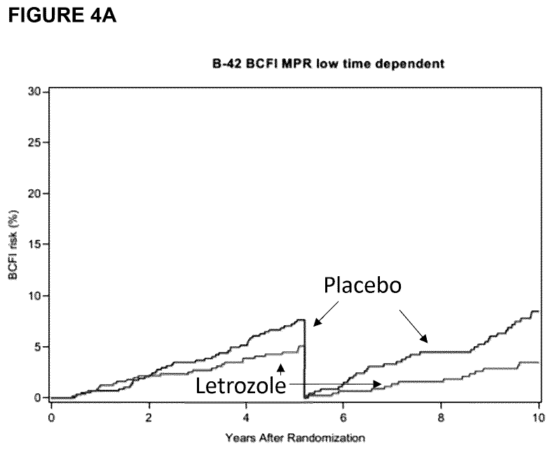

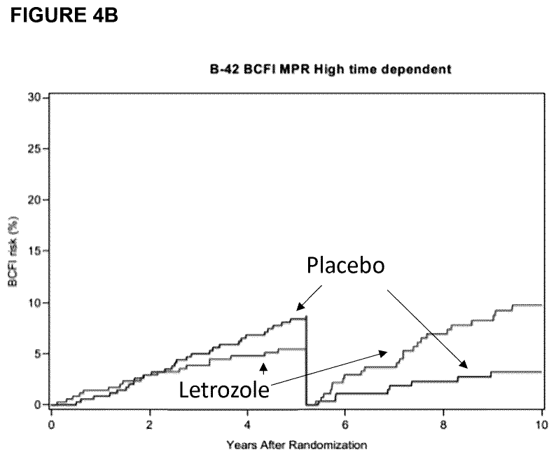

For BCFI, the assumption of hazards proportionality between treatment groups was not satisfied for the MP-H subgroup. Based on the MP-H subgroup a change point of 5.2 years was identified. The effect of EET was not different between MP-H (HR=0.65, 95% CI 0.36-1.17, p=0.15) and MP-L (HR=0.65, 95% CI 0.40-1.06, p=0.08) subgroups prior to 5.2 years, however the difference in the EET effect was statistically significantly different after 5.2 years (treatment-by-MP risk group interaction p=0.006): MP-H (HR=2.77, 95% CI 1.28-5.99, p=0.007) and MP-L (HR=0.37, 95% CI 0.21-0.66, p<0.001) (FIG. 4).

TABLE 3
Distribution of demographic, clinical and treatment characteristics of patients
in NSABP B-42 according to the inclusion in the translational MP sub-cohort.

| | Translational MP | | Excluded B-42 | | | Overall B-42 | |
|---|---|---|---|---|---|---|---|
| | sub-cohort (n = 1,866) | | cohort (n = 2,037) | | | population (n = 3,903) | |
| Characteristic | n | % | n | % | p-v | n | % |
| Age at randomization, yrs | | | | | 0.19 | | |
| <60 | 623 | 33.4 | 721 | 35.4 | | 1344 | 34.4 |
| >=60 | 1243 | 66.6 | 1316 | 64.6 | | 2559 | 65.6 |
| Pathologic Nodal Status | | | | | 0.25 | | |
| Negative | 1053 | 56.4 | 1187 | 58.3 | | 2240 | 57.4 |
| Positive | 813 | 43.6 | 850 | 41.7 | | 1663 | 42.6 |
| Lowest BMD T-score | | | | | 0.34 | | |
| ≤−2.0 | 469 | 25.1 | 485 | 23.8 | | 954 | 24.4 |
| >−2.0 | 1397 | 74.9 | 1552 | 76.2 | | 2949 | 75.6 |
| Received prior tamoxifen | | | | | 0.81 | | |
| No | 1140 | 61.1 | 1237 | 60.7 | | 2377 | 60.9 |
| Yes | 726 | 38.9 | 800 | 39.3 | | 1526 | 39.1 |
| HER2 Status | | | | | 0.04 | | |
| Positive | 262 | 14.0 | 297 | 14.6 | | 559 | 14.3 |
| Negative | 1479 | 79.3 | 1562 | 76.7 | | 3041 | 77.9 |
| Not done/Unknown | 125 | 6.7 | 178 | 8.7 | | 303 | 7.8 |
| Surgery Type | | | | | 0.36 | | |
| Lumpectomy | 1121 | 60.1 | 1253 | 61.5 | | 2374 | 60.8 |
| Mastectomy | 745 | 39.9 | 784 | 38.5 | | 1529 | 39.2 |
| Treatment | | | | | 0.30 | | |
| Placebo | 950 | 50.9 | 1003 | 49.2 | | 1953 | 50.0 |
| Letrozole | 916 | 49.1 | 1034 | 50.8 | | 1950 | 50.0 |

TABLE 4
The EET effect in the subgroups of patients defined by MP-risk categories.

| | | Letrozole | | Placebo | | | | | |
|---|---|---|---|---|---|---|---|---|---|
| | MP | | 10-year | | 10-year | Abs. | | | |
| | Risk | | event | | event | benefit | HR | | |
| Endpoint | Group | # Events | rate (%) | # Events | rate (%) | (%) | (95% CI) | p | P interaction |
| DR | L | 20 | 3.5 | 43 | 7.2 | 3.7 | 0.43 | 0.002 | 0.38 |
| | | | | | | | (0.25, 0.74) | | |
| | H | 15 | 4.9 | 24 | 7.3 | 2.4 | 0.65 | 0.19 | |
| | | | | | | | (0.34, 1.24) | | |
| DFS | L | 113 | 79.7 | 165 | 71.9 | 7.8 | 0.67 | <0.001 | 0.015 |
| | | | | | | | (0.52, 0.85) | | |
| | H | 91 | 71.2 | 88 | 72.8 | −1.6 | 1.10 | 0.55 | |
| | | | | | | | (0.82, 1.47) | | |
| BCFI | L | 44 | 8.4 | 83 | 15.4 | 7.0 | 0.51 | <0.001 | 0.006 |
| | | | | | | | (0.35, 0.74) | | |
| | H | 42 | 14.6 | 38 | 11.6 | −3.0 | 1.15 | 0.53 | |
| | | | | | | | (0.74, 1.79) | | |

Number of patients in MP-H (P: 354, L: 352), MP-L (P: 596, L: 564)

TABLE 5
The EET effect in the subgroups of patients defined by MP-risk categories.

| | | Letrozole | | Placebo | | | | | |
|---|---|---|---|---|---|---|---|---|---|
| | MP | | 10-year | | 10-year | Abs. | | | |
| | Risk | | event | | event | benefit | HR | | |
| Endpoint | Group | # Events | rate (%) | # Events | rate (%) | (%) | (95% CI) | p | P interaction |
| DR | UL | 3 | 2.9 | 6 | 5.8 | 3.0 | 0.53 | 0.37 | 0.69 |
| | | | | | | | (0.13, 2.15) | | |
| | LNUL | 17 | 3.6 | 37 | 7.6 | 4.0 | 0.42 | 0.003 | |
| | | | | | | | (0.23, 0.76) | | |
| | H | 15 | 4.9 | 24 | 7.3 | 2.4 | 0.65 | 0.19 | |
| | | | | | | | (0.34, 1.24) | | |
| DFS | UL | 20 | 82.5 | 26 | 80.7 | 1.8 | 0.82 | 0.50 | 0.042 |
| | | | | | | | (0.45, 1.48) | | |
| | LNUL | 93 | 78.9 | 139 | 69.4 | 9.5 | 0.64 | <0.001 | |
| | | | | | | | (0.49, 0.83) | | |
| | H | 91 | 71.2 | 88 | 72.8 | −1.6 | 1.10 | 0.55 | |
| | | | | | | | (0.82, 1.47) | | |
| BCFI | UL | 8 | 7.3 | 13 | 11.4 | 4.1 | 0.67 | 0.38 | 0.02 |
| | | | | | | | (0.28, 1.65) | | |
| | LNUL | 36 | 8.7 | 70 | 16.6 | 7.9 | 0.48 | <0.001 | |
| | | | | | | | (0.32, 0.73) | | |
| | H | 42 | 14.6 | 38 | 11.6 | −3.0 | 1.15 | 0.53 | |
| | | | | | | | (0.74, 1.79) | | |

Number of patients in MP-H (P: 354, L: 352), MP-L (P: 596, L: 564), MP-UL (P: 133, L: 119), MP-LNUL (P: 463, L: 445)

### INCORPORATION OF SEQUENCE LISTING

Incorporated herein by reference in its entirety is the Sequence Listing for the above-identified application conforming to the rules of WIPO Standard ST.25. The Sequence Listing is disclosed on a computer-readable ASCII formatted text file (.txt), entitled “Sequence_Listing.294-637_PCTUS.txt”, created on Oct. 31, 2023, and is 44 KB in size.

What is claimed is:

1. A method of treating a hormone receptor (HR)-positive breast cancer in an individual, comprising administering endocrine therapy to the individual, wherein said cancer is typed as having a low risk of recurrence and wherein said endocrine therapy is administered for more than five years.

2. The method according to claim 1, wherein the endocrine therapy comprises an aromatase inhibitor and/or anti-oestrogen therapy.

3. The method according to claim 1, wherein the endocrine therapy comprises letrozole and/or tamoxifen.

4. The method according to claim 1, wherein the endocrine therapy has a duration of at least six years.

5. The method according to claim 1, wherein the cancer is typed using MammaPrint.

6. The method according to claim 1, wherein the cancer is considered as having low risk of recurrence if it is typed as MammaPrint low risk (MP-L).

7. The method according to claim 1, wherein the individual is a post-menopausal woman.

8. The method according to claim 1, wherein the cancer is not typed as MammaPrint ultralow risk (MP-UL).

9. A method of treating an individual with hormone receptor (HR)-positive breast cancer with endocrine therapy administered for more than five years if said cancer is typed as having low risk of recurrence and treating an individual with HR-positive breast cancer with endocrine therapy administered for five years or less if said cancer is typed as having high risk of recurrence.

10. The method of treating according to claim 9, wherein the endocrine therapy administered for more than five years comprises an aromatase inhibitor and/or anti-oestrogen therapy.

11. The method of treating according to claim 9, wherein the endocrine therapy administered for more than five years comprises letrozole and/or tamoxifen.

12. The method of treating according to claim 9, wherein the endocrine therapy administered for five years or less comprises an aromatase inhibitor and/or anti-oestrogen therapy.

13. The method of treating according to claim 9, wherein the endocrine therapy administered for more than five years has a duration of at least six year.

14. The method of treating according to claim 9, wherein the cancer is typed using MammaPrint.

15. The method of treating according to claim 9, wherein the cancer is considered as having low risk of recurrence if they are typed as MammaPrint low risk (MP-L) and wherein the cancer is considered as having high risk of recurrence if they are typed as MammaPrint high risk (MP-H).

16. The method of treating according to claim 9, wherein the individual is a post-menopausal woman

17. The method of treating according to claim 9, wherein the individual with HR-positive breast cancer is treated with endocrine therapy administered for five years or less if said cancer is typed as having MammaPrint ultralow risk (MP-UL).


SEQUENCE LISTING (231 sequences)

SEQ ID NO: 1 - DNA, 60 bp, Artificial Sequence
  MammaPrint probe
ctgagtggtcagagatctgtaaagcatgactttcaaggatggttcttaggggactgtgta

SEQ ID NO: 2 - DNA, 60 bp, Artificial Sequence
  MammaPrint probe
aggacttgaatgaggaaaccaacactttgagaaaccaaagtcctttttcccaaaggttct

SEQ ID NO: 3 - DNA, 60 bp, Artificial Sequence
  MammaPrint probe
gccattaagatttggatgggaagttatgggtaatgagaatataatgacatcttgcaacat

SEQ ID NO: 4 - DNA, 60 bp, Artificial Sequence
  MammaPrint probe
gatggcccagcctgtaagatactgtatatgcgctgctgtagataccggaatgaattttct

SEQ ID NO: 5 - DNA, 60 bp, Artificial Sequence
  MammaPrint probe
ggcctcacattctgctctgctaagtttggagaaaacagaacaataaaccagatgcaggtg

SEQ ID NO: 6 - DNA, 60 bp, Artificial Sequence
  MammaPrint probe
aagtactggaatgtaatggttgaaattcctattcagtgatctggaagaactctaatgttc

SEQ ID NO: 7 - DNA, 60 bp, Artificial Sequence
  MammaPrint probe
ccaacgcacaccagtcttctcaatctgactgtaatctaatctgttgtgcttttgttggac

SEQ ID NO: 8 - DNA, 60 bp, Artificial Sequence
  MammaPrint probe
ggtttaaagctgaagaggttgaagctaaaaggaaaaggttgttgttaatgaatatcaggc

SEQ ID NO: 9 - DNA, 60 bp, Artificial Sequence
  MammaPrint probe
ggctaaaagggaaaaaggatatgtggagaatcatcaagatatgaattgaatcgctgcgat

SEQ ID NO: 10 - DNA, 60 bp, Artificial Sequence
  MammaPrint probe
cctttcaaacatgatcaaagatttcccaatgtgatctcatcatcatggatactcaatttg

SEQ ID NO: 11 - DNA, 60 bp, Artificial Sequence
  MammaPrint probe
ggggaacaatgagggcatttcatgaaccatctcaggcacttctgcatcacggaagacctg

SEQ ID NO: 12 - DNA, 60 bp, Artificial Sequence
  MammaPrint probe
tgccttgagaatttcaaaagaggtaatcaggaaaagagagagagaaaaactacacgctgt

SEQ ID NO: 13 - DNA, 60 bp, Artificial Sequence
  MammaPrint probe
gtctgggattaagggcaaatctattacttttgcaaactgtcctctacatcaattaacatc

SEQ ID NO: 14 - DNA, 60 bp, Artificial Sequence
  MammaPrint probe
taaaaaagaaatagtcagtgttttcctcctttcaaccgagactatttctggattgtgtgc

SEQ ID NO: 15 - DNA, 60 bp, Artificial Sequence
  MammaPrint probe
ttttcagaaagaagtctggaccaggctgaaggcatttgcaaagcttcccccaaatgtctt

SEQ ID NO: 16 - DNA, 60 bp, Artificial Sequence
  MammaPrint probe
cctcattgccttattcggagtactattatccaatatatgaaatcaaagattgtctcctga

SEQ ID NO: 17 - DNA, 60 bp, Artificial Sequence
  MammaPrint probe
tggcatcatacaaagagcaggagaagcaaacacccagaactcttttgctggtcagagatt

SEQ ID NO: 18 - DNA, 60 bp, Artificial Sequence
  MammaPrint probe
tccagacctaccttgtacgcacatagacattttcatatgcactggatggagttagggaaa

SEQ ID NO: 19 - DNA, 60 bp, Artificial Sequence
  MammaPrint probe
atctttgttaattattttggggagtagttgggaaatggaaaggtgaattggctctagagg

SEQ ID NO: 20 - DNA, 60 bp, Artificial Sequence
  MammaPrint probe
gttcatttccagccctttctagatctgatcttttagggggaaagacagcttaaaatgttc

SEQ ID NO: 21 - DNA, 60 bp, Artificial Sequence
  MammaPrint probe
tgaatgtcatgtttatgtcatagacgtagaaaacgcatccttgaattaaactgccttaac

SEQ ID NO: 22 - DNA, 60 bp, Artificial Sequence
  MammaPrint probe
tactggagtaactgagtcgggacgctgaatctgaatccaccaataaataaagcttctgca

SEQ ID NO: 23 - DNA, 60 bp, Artificial Sequence
  MammaPrint probe
cagattccccagaaactaccttttgcccaaagaacatgctcagtatttggggcatttcct

SEQ ID NO: 24 - DNA, 60 bp, Artificial Sequence
  MammaPrint probe
aggcaggggtggtgattcatgctgtgtgactgactgtgggtaataaacacacctgtcccc

SEQ ID NO: 25 - DNA, 60 bp, Artificial Sequence
  MammaPrint probe
tggatttctaaactgctcaattttgactcaaaggtgctatttaccaaacactctccctac

SEQ ID NO: 26 - DNA, 60 bp, Artificial Sequence
  MammaPrint probe
ccaatccaacaactataggctgggttaaataaaaggtcattattgtctatattccaagtg

SEQ ID NO: 27 - DNA, 60 bp, Artificial Sequence
  MammaPrint probe
tctaccacattaaattctccattacatctcactattggtaatggcttaagtgtaaagagc

SEQ ID NO: 28 - DNA, 60 bp, Artificial Sequence
  MammaPrint probe
tgaggaattcttgtacgcagttttctttggctttacgagccgattaaaagaccgtgtgaa

SEQ ID NO: 29 - DNA, 60 bp, Artificial Sequence
  MammaPrint probe
ctggtctttgaaagaaatgtactactaaagagcactagttgtgaatttagggtgttaaac

SEQ ID NO: 30 - DNA, 60 bp, Artificial Sequence
  MammaPrint probe
atgatgggagagctctggcagatgtcccaatcctggaggtcatccattaggaattaaatt

SEQ ID NO: 31 - DNA, 60 bp, Artificial Sequence
  MammaPrint probe
caacttgctctttcatatgagttggtcatagcatgtaagaaccaatcttgaaatatcgtt

SEQ ID NO: 32 - DNA, 60 bp, Artificial Sequence
  MammaPrint probe
cctggatcagagtaagaatgtcttaagaagaggtttgtaaggtcttcataacaaagtggt

SEQ ID NO: 33 - DNA, 60 bp, Artificial Sequence
  MammaPrint probe
ctcctggactgcttcttttggctctccgacaactccggccaataaacactttctgaattg

SEQ ID NO: 34 - DNA, 60 bp, Artificial Sequence
  MammaPrint probe
aaccaacccataattgcattttacttgtcgtggttcgatctgattgtattgtcgaaggac

SEQ ID NO: 35 - DNA, 60 bp, Artificial Sequence
  MammaPrint probe
attcctttatgagctctccatatccttcttgagaaactggttaaaaaaggaataggggta

SEQ ID NO: 36 - DNA, 60 bp, Artificial Sequence
  MammaPrint probe
agtgggggttgtgtaaaggggaagtcatcttttgagatccagatagacatggtttgtgca

SEQ ID NO: 37 - DNA, 60 bp, Artificial Sequence
  MammaPrint probe
tcagcttaagtacttattgtggtagtgagtcctacggtatttcagtaaaaaggaattcat

SEQ ID NO: 38 - DNA, 60 bp, Artificial Sequence
  MammaPrint probe
ggcaagagttatcatagaacaacaaaatagagtggactcttttagagcatctatatctgc

SEQ ID NO: 39 - DNA, 60 bp, Artificial Sequence
  MammaPrint probe
ggagtttctgtttagggcattaaaaattcccgcaaactataaagagcaatgttttcagtc

SEQ ID NO: 40 - DNA, 60 bp, Artificial Sequence
  MammaPrint probe
ataattctctgtacaggggggtttgtgctatacactgggatgtctaattgcagcaataaa

SEQ ID NO: 41 - DNA, 60 bp, Artificial Sequence
  MammaPrint probe
aggactttaatcttggtgatgccttggacagcagcaactccatgcaaaccatccaaaaga

SEQ ID NO: 42 - DNA, 60 bp, Artificial Sequence
  MammaPrint probe
gggcaaaatgtatcactccaaacactactgattcagcattgttttcatgtcttaaaattg

SEQ ID NO: 43 - DNA, 60 bp, Artificial Sequence
  MammaPrint probe
ctggatgtttagcttcttactgcaaaaacataagtaaaacagtcaactttaccatttccg

SEQ ID NO: 44 - DNA, 60 bp, Artificial Sequence
  MammaPrint probe
ggtaacttgcaggaatattctattggaaaagataacaggaagtacaagtgcttcttgacc

SEQ ID NO: 45 - DNA, 60 bp, Artificial Sequence
  MammaPrint probe
tcaatggttagcagaagggagaaaagaaagcaggaaaatgtgctattgagattccagtgg

SEQ ID NO: 46 - DNA, 60 bp, Artificial Sequence
  MammaPrint probe
cctgggtttacaacgctgttaggaaaattaaccaatgaataaagcaacgttcagtgcgca

SEQ ID NO: 47 - DNA, 60 bp, Artificial Sequence
  MammaPrint probe
tttttgtaccttgtcactataactacttcctagtcaaagaacgaaatgtaactgttaccg

SEQ ID NO: 48 - DNA, 60 bp, Artificial Sequence
  MammaPrint probe
ttctagctgttattttgctatttggcatttacataaaagcacacgatgaagcaggtatcg

SEQ ID NO: 49 - DNA, 60 bp, Artificial Sequence
  MammaPrint probe
ttgggtttatttccaggtcacagaattgctgttaacactagaaaacacacttcctgcacc

SEQ ID NO: 50 - DNA, 60 bp, Artificial Sequence
  MammaPrint probe
gaacagctccttactctgaggaagttgattcttatttgatggtggtattgtgaccactga

SEQ ID NO: 51 - DNA, 60 bp, Artificial Sequence
  MammaPrint probe
ctttcttatttactaagaatttgcctgtttgaataagaacaaaacgctaaggtgggtagc

SEQ ID NO: 52 - DNA, 60 bp, Artificial Sequence
  MammaPrint probe
ctagagagcagaaataaaaagcatgactatttccaccatcaaatgctgtagaatgcttgg

SEQ ID NO: 53 - DNA, 60 bp, Artificial Sequence
  MammaPrint probe
gttcaggggcatcacctactttgcttacttgattcaaggctctcattaaagacattttag

SEQ ID NO: 54 - DNA, 60 bp, Artificial Sequence
  MammaPrint probe
gttggtagagggagtatgataaaatgtttaaatctcatttggttaccttgagtcctggaa

SEQ ID NO: 55 - DNA, 60 bp, Artificial Sequence
  MammaPrint probe
aattcaacagtgtggaagctttaggggaacatggagaaagaaggagaccacataccccaa

SEQ ID NO: 56 - DNA, 60 bp, Artificial Sequence
  MammaPrint probe
caagttgtgcaaagtgagaaagatctttgtgggcacaaaaggaatccctcatctggtgac

SEQ ID NO: 57 - DNA, 60 bp, Artificial Sequence
  MammaPrint probe
caagagaacctggagaaaactaccgtattcaagagattaatcaaaatcagtgttttagcc

SEQ ID NO: 58 - DNA, 60 bp, Artificial Sequence
  MammaPrint probe
ccgaatgaccttaaaggtgatcggctttaacgaatatgtttacatatgcatagcgctgca

SEQ ID NO: 59 - DNA, 60 bp, Artificial Sequence
  MammaPrint probe
agtttatgggccagaatattctgtataccagacattggtaagctctcatggtttacagga

SEQ ID NO: 60 - DNA, 60 bp, Artificial Sequence
  MammaPrint probe
ccatgtggccagtctaccatggggcccaggagttggggaaacacaataaaggtggcatac

SEQ ID NO: 61 - DNA, 60 bp, Artificial Sequence
  MammaPrint probe
atgcttaaacccacggaagggggagactctttcggatttgtagggtgaaatggcaattat

SEQ ID NO: 62 - DNA, 60 bp, Artificial Sequence
  MammaPrint probe
ttctttcttcaaagagtcatcagaataacatggattgaagagacttccgaacacttgcta

SEQ ID NO: 63 - DNA, 60 bp, Artificial Sequence
  MammaPrint probe
tgaagtcagcgttaaccatgtgcatacaacttaaggaattttttcctcctcatgtaaatt

SEQ ID NO: 64 - DNA, 60 bp, Artificial Sequence
  MammaPrint probe
gttaaacagggattatagtacttgtctcacaaagtttctgtgagaattaaacaaggggat

SEQ ID NO: 65 - DNA, 60 bp, Artificial Sequence
  MammaPrint probe
cagcctgtgtgatacaagtttgatcccaggaacttgagttctaagcagtgctcgtgaaaa

SEQ ID NO: 66 - DNA, 60 bp, Artificial Sequence
  MammaPrint probe
aatgcacagatctgcttgatcaattcccttgaatagggaagtaacatttgccttaaattt

SEQ ID NO: 67 - DNA, 60 bp, Artificial Sequence
  MammaPrint probe
tttccaataaccacctaaattttaacaaaggttccttctaagtggtagaacttggggtgg

SEQ ID NO: 68 - DNA, 60 bp, Artificial Sequence
  MammaPrint probe
agttatgcttcccttcatgttatatgcacattgccaagaattactgtcaagagaaatgat

SEQ ID NO: 69 - DNA, 60 bp, Artificial Sequence
  MammaPrint probe
aaggtttgaaggttacggctcagggctgccccattaaagtcagtgttgtgttctaaaaaa

SEQ ID NO: 70 - DNA, 60 bp, Artificial Sequence
  MammaPrint probe
ggactgtatgaatttatagaaaattgaatctaatttcagaagagcgcactgtcttctcag

SEQ ID NO: 71 - DNA, 60 bp, Artificial Sequence
  MammaPrint probe
tacatttctttgggtttctagagacgcccctaagtcacctgcttcattagacggtttcca

SEQ ID NO: 72 - DNA, 60 bp, Artificial Sequence
  MammaPrint probe
ggcctaattgagggaaggaggaaattcataccagcagttttcaaataaaagaattgttct

SEQ ID NO: 73 - DNA, 60 bp, Artificial Sequence
  MammaPrint probe
tccaattctacactcagttaaagaccattacttctcagtggaaagaagaagatgctactc

SEQ ID NO: 74 - DNA, 60 bp, Artificial Sequence
  MammaPrint probe
gtggggacttcgtgggaggcactcatggctctctgggtctaatgaataaagtcctccaca

SEQ ID NO: 75 - DNA, 60 bp, Artificial Sequence
  MammaPrint probe
ccaggatcttaaggaagaatattctaggaagaaggaaactatttctactgctaataaagc

SEQ ID NO: 76 - DNA, 60 bp, Artificial Sequence
  MammaPrint probe
agaaaacccttttctacagttagggttgagttacttcctatcaagccagtacgtgctaac

SEQ ID NO: 77 - DNA, 60 bp, Artificial Sequence
  MammaPrint probe
tagggaatgaatgaatgaatatggattgctgttaactagaaacacttctgtatgtcagtc

SEQ ID NO: 78 - DNA, 60 bp, Artificial Sequence
  MammaPrint probe
gtacttagctggaagaacatgttaattctgcaatatgtttcttggttaaacattgcacag

SEQ ID NO: 79 - DNA, 60 bp, Artificial Sequence
  MammaPrint probe
actctcttaggtcatttttcaatgtgtgtaaccaaaagttaatcagaataaagcggaagc

SEQ ID NO: 80 - DNA, 60 bp, Artificial Sequence
  MammaPrint probe
aatgctttgttggagtttaaaaattcagggaaaaaatcggcagaccattagttactatgg

SEQ ID NO: 81 - DNA, 60 bp, Artificial Sequence
  MammaPrint probe
aagaaaccagcatgtgactttcctagataacactgctttctcataataaagactatttgc

SEQ ID NO: 82 - DNA, 60 bp, Artificial Sequence
  MammaPrint probe
gttggcattgatatggtacaacctgcaaattacttgcagttctgagtttcagataaaaca

SEQ ID NO: 83 - DNA, 60 bp, Artificial Sequence
  MammaPrint probe
agtgtcattttaagggacatttttatgacttttatgtgtatgtttatgtagaaatttgga

SEQ ID NO: 84 - DNA, 60 bp, Artificial Sequence
  MammaPrint probe
actcacttcttttcaggtgtagctacaattgtgtaatgtacaatattagagaaaggacag

SEQ ID NO: 85 - DNA, 60 bp, Artificial Sequence
  MammaPrint probe
cctgggagcaaatgaacaatagctaagtgtcttggtatttaaagagtaaattatttgtgg

SEQ ID NO: 86 - DNA, 60 bp, Artificial Sequence
  MammaPrint probe
ccaagaatatatgctacagatataagacagacatggtttggtcctatatttctagtcatg

SEQ ID NO: 87 - DNA, 60 bp, Artificial Sequence
  MammaPrint probe
atgctttcctaaatcagatgttttggtcaagtagtttgactcagtataggtagggagata

SEQ ID NO: 88 - DNA, 60 bp, Artificial Sequence
  MammaPrint probe
atttgtgtggacaaaaatatttacacttagggtttggagctattcaagaggaaatgtcac

SEQ ID NO: 89 - DNA, 60 bp, Artificial Sequence
  MammaPrint probe
aaatatactatgtttgcgaaccttggtagctatgatgagagctattatcatctgtggtgg

SEQ ID NO: 90 - DNA, 60 bp, Artificial Sequence
  MammaPrint probe
tcaatgaaagttcaagaacctcctgtacttaaacacgattcgcaacgttctgttattttt

SEQ ID NO: 91 - DNA, 60 bp, Artificial Sequence
  MammaPrint probe
accttgatagttcaccacgtctgatggatccctgttttaaataaaaacgattcactttaa

SEQ ID NO: 92 - DNA, 60 bp, Artificial Sequence
  MammaPrint probe
taaaatacttcaatcctggattcacagtgggaacaagtttctattaaaaggcaaatgctg

SEQ ID NO: 93 - DNA, 60 bp, Artificial Sequence
  MammaPrint probe
ggctgtgaacaatgttaaatagcatcagtttgtccaatagttttaaaggccataatcatc

SEQ ID NO: 94 - DNA, 60 bp, Artificial Sequence
  MammaPrint probe
acgagtaccggcatgttatgttacccagagaactttccaaacaagtacctaaaactcatc

SEQ ID NO: 95 - DNA, 60 bp, Artificial Sequence
  MammaPrint probe
attttttagaaaatacacacttttcaggagaaacctgagcatgattttggattctccacc

SEQ ID NO: 96 - DNA, 60 bp, Artificial Sequence
  MammaPrint probe
cagctcagaccatttcctaatcagttgaaagggaaacaagtatttcagtctcaaaattga

SEQ ID NO: 97 - DNA, 60 bp, Artificial Sequence
  MammaPrint probe
cactgcagactctcaagagatcaatcaaattgccagaaacagtttggtttttcatatgga

SEQ ID NO: 98 - DNA, 60 bp, Artificial Sequence
  MammaPrint probe
tgaaactttcttctgatgagtttctttaacgtacaggatggagtaaaacaaatggtacag

SEQ ID NO: 99 - DNA, 60 bp, Artificial Sequence
  MammaPrint probe
caattcttgagagttaatgtgatcatgatattgcaaacaactataaatggtctctaggcc

SEQ ID NO: 100 - DNA, 60 bp, Artificial Sequence
  MammaPrint probe
gaaggaaacaccgagtctctgtataatctatttacataaaatgggtgatatgcgaacagc

SEQ ID NO: 101 - DNA, 60 bp, Artificial Sequence
  MammaPrint probe
agcaacctgggccttgtactgtctgtgtttttaaaaccactaaagtgcaagaattacatt

SEQ ID NO: 102 - DNA, 60 bp, Artificial Sequence
  MammaPrint probe
gggaatttgatgcagtaaagattaccctgttttatgattgttccttgaaagtcaaatggg

SEQ ID NO: 103 - DNA, 60 bp, Artificial Sequence
  MammaPrint probe
taaggctaatgataccaatgagggttggtttattatcaaacctgaatagctgtggtttct

SEQ ID NO: 104 - DNA, 60 bp, Artificial Sequence
  MammaPrint probe
tggggagatacatcttatagagttagaaatagaatctgaatttctaaagggagattctgg

SEQ ID NO: 105 - DNA, 60 bp, Artificial Sequence
  MammaPrint probe
tatcttgaaactgaccaaacgcttattgtgtaagataaaccagttgaatcattgaggatc

SEQ ID NO: 106 - DNA, 60 bp, Artificial Sequence
  MammaPrint probe
ttctctgaaggaatcatgttcagtgttcgaccacctaagaaaagttggaaaaagatcttc

SEQ ID NO: 107 - DNA, 60 bp, Artificial Sequence
  MammaPrint probe
tgtcatagacatgtattggggagcttccaattagcatacatagacacatgtgtcagtggc

SEQ ID NO: 108 - DNA, 60 bp, Artificial Sequence
  MammaPrint probe
tgtccatgctacaagaagttatgagccttgttctaagtacagatgaaccttgtatttgtg

SEQ ID NO: 109 - DNA, 60 bp, Artificial Sequence
  MammaPrint probe
atcccgatttcagtcagacaaatactcatttcagagattctatacttcatggaatcaaga

SEQ ID NO: 110 - DNA, 60 bp, Artificial Sequence
  MammaPrint probe
agttactttcttaatgtgacctagcaataggcatagctacgtggcactatattctggcca

SEQ ID NO: 111 - DNA, 60 bp, Artificial Sequence
  MammaPrint probe
gaaatctctctacacagatgagtcatccaaacctgggaaaaaataaaagaactgcaatca

SEQ ID NO: 112 - DNA, 60 bp, Artificial Sequence
  MammaPrint probe
aaattgctaagtggaatgcatgaattgcattatgttctctggtaacacgtagagttcaga

SEQ ID NO: 113 - DNA, 60 bp, Artificial Sequence
  MammaPrint probe
ccaaaggtcttggtacaaccagctgcccattttgtgaaatttttatgtagaataaacatt

SEQ ID NO: 114 - DNA, 60 bp, Artificial Sequence
  MammaPrint probe
gtttcgggtctttacctcatagtatgaaattagtaagacactgcatagattttgccctga

SEQ ID NO: 115 - DNA, 60 bp, Artificial Sequence
  MammaPrint probe
gagtacggatgggaaactattgtgcacaagtctttccagaggagtttcttaatgagatat

SEQ ID NO: 116 - DNA, 60 bp, Artificial Sequence
  MammaPrint probe
tattttatcagcactttatgcacgtattattgacattaatacctaatcggcgagtgccca

SEQ ID NO: 117 - DNA, 60 bp, Artificial Sequence
  MammaPrint probe
tgccctatggaaaacttgtccaaataacatttcttgaacaataggagaacagctaaattg

SEQ ID NO: 118 - DNA, 60 bp, Artificial Sequence
  MammaPrint probe
ctccttgtcattgaccttagctaaaccatggcaattcataaatagaggaaacattaatga

SEQ ID NO: 119 - DNA, 60 bp, Artificial Sequence
  MammaPrint probe
ctgaacgagaacaagaatcagaagaagaaatgtgactttgatgagcttccagtttttcta

SEQ ID NO: 120 - DNA, 60 bp, Artificial Sequence
  MammaPrint probe
tatattatcagtctgtaccagtagaccagtaccctaactactgaaaagaatatggcagtt

SEQ ID NO: 121 - DNA, 60 bp, Artificial Sequence
  MammaPrint probe
agtaacgctaactttgtacggacgatgtctcatggattaaataatattctttatggcagt

SEQ ID NO: 122 - DNA, 60 bp, Artificial Sequence
  MammaPrint probe
gtggatctacctcagttaaacagttgggtgctattactaagtctgtcaaattaaattgga

SEQ ID NO: 123 - DNA, 60 bp, Artificial Sequence
  MammaPrint probe
cattctaaagggaaatcagtaaaatgtcttgataattggtatccaaatcacttgtgtgcc

SEQ ID NO: 124 - DNA, 60 bp, Artificial Sequence
  MammaPrint probe
ccaaagacaaacgattagaagatggctatttcagaataggaaaatttgagaatggtgttg

SEQ ID NO: 125 - DNA, 60 bp, Artificial Sequence
  MammaPrint probe
caaacttcctgacactacttccatatttgcactaaaggagattcagctacaaaaggaggc

SEQ ID NO: 126 - DNA, 60 bp, Artificial Sequence
  MammaPrint probe
accttcctatgaagatcatggaatcaaatacgggacattgaactaatacttggactttga

SEQ ID NO: 127 - DNA, 60 bp, Artificial Sequence
  MammaPrint probe
ggctaacacaatctaattttggtttaagagacaaatctagagtctcaaatgatctcagag

SEQ ID NO: 128 - DNA, 60 bp, Artificial Sequence
  MammaPrint probe
tggacccttaaatatgactaaaatcacagcaatattgttacatacgggttatatgccaac

SEQ ID NO: 129 - DNA, 60 bp, Artificial Sequence
  MammaPrint probe
taagcattgtgaaggaagattaatatagccaaataactagagtgatcagttctaccagag

SEQ ID NO: 130 - DNA, 60 bp, Artificial Sequence
  MammaPrint probe
cctggataaaagtactgtatgattttgtgatggatgatacaataagtccctactcaagaa

SEQ ID NO: 131 - DNA, 60 bp, Artificial Sequence
  MammaPrint probe
gctttgttactttgttaggtacgaatcacataagggagattgtatacaagttggagcaat

SEQ ID NO: 132 - DNA, 60 bp, Artificial Sequence
  MammaPrint probe
taaaagatgaagaaagctattaggtatatttgtacatgactgcaaatgagtctatgcccg

SEQ ID NO: 133 - DNA, 60 bp, Artificial Sequence
  MammaPrint probe
gtgtgttatctttatatgtcaaactggttgaacactgtaatgagaataaactgcacagag

SEQ ID NO: 134 - DNA, 60 bp, Artificial Sequence
  MammaPrint probe
gattattgtacgaagtgtctctgtaattatcatactactaaagactgttcagatggcaag

SEQ ID NO: 135 - DNA, 60 bp, Artificial Sequence
  MammaPrint probe
catttgtattaatggaatactaagtccctctgtgatttctgaaccaagctattcctaggc

SEQ ID NO: 136 - DNA, 60 bp, Artificial Sequence
  MammaPrint probe
atgaagagatttctcaagctattcttgatttcagaaacgcaaaaaatgggtttgaaaggg

SEQ ID NO: 137 - DNA, 60 bp, Artificial Sequence
  MammaPrint probe
agccaatcatgagtacgtaaagtgatttttgctctctgtgtacaacttttaaaatctgac

SEQ ID NO: 138 - DNA, 60 bp, Artificial Sequence
  MammaPrint probe
atcctagaccatattttcaagtcatcttagcagctaggattctcaaatggaagtgttata

SEQ ID NO: 139 - DNA, 60 bp, Artificial Sequence
  MammaPrint probe
agtgatttcatgctagaaaaattggaaactaaaagtgtgtagctaggttatttcggagtg

SEQ ID NO: 140 - DNA, 60 bp, Artificial Sequence
  MammaPrint probe
gctaagccaagtagtagcagtaaaacttctgatcctctagcatcaaaaactacaactaca

SEQ ID NO: 141 - DNA, 60 bp, Artificial Sequence
  MammaPrint probe
ggaaagaagttgaaagcatcttgaagaaaaactcagattggatatgggattggtcaagtc

SEQ ID NO: 142 - DNA, 60 bp, Artificial Sequence
  MammaPrint probe
acctggatatgtctgtgaggctcctgaaaggagacaaataaagtcaatatatttgcacaa

SEQ ID NO: 143 - DNA, 60 bp, Artificial Sequence
  MammaPrint probe
gggtatgaaagatgagtgtctgtaaaaatccttcttagaaatgtatttcctcaagactct

SEQ ID NO: 144 - DNA, 60 bp, Artificial Sequence
  MammaPrint probe
cagatggcaagattgagtttatttcaacaatggaaggatataagtatccagtatatggtg

SEQ ID NO: 145 - DNA, 60 bp, Artificial Sequence
  MammaPrint probe
gaaactgtgtcaccctaaagaagcatataatcatagcattaaaaatgcacacattactcc

SEQ ID NO: 146 - DNA, 60 bp, Artificial Sequence
  MammaPrint probe
caagcgtgtttctagagaacagttgagagagaatctcaagattctacttggtggtttgct

SEQ ID NO: 147 - DNA, 60 bp, Artificial Sequence
  MammaPrint probe
ccgacaagaggagatcattttagatattaccgaaatgaagaaagcttgcaattagtgaac

SEQ ID NO: 148 - DNA, 60 bp, Artificial Sequence
  MammaPrint probe
taatagcaaaatttaacccgttactctttaaccttgtactggaaattctaagcagtgcag

SEQ ID NO: 149 - DNA, 60 bp, Artificial Sequence
  MammaPrint probe
cttcctacctctggtgatggtttccacaggaacaacagcatctttcaccaagatgggtgg

SEQ ID NO: 150 - DNA, 60 bp, Artificial Sequence
  MammaPrint probe
aaatcattcggtaaatccaaactgctatgcaaaagttatgatggttaacggtgatcacag

SEQ ID NO: 151 - DNA, 60 bp, Artificial Sequence
  MammaPrint probe
ttgggtttctagtcctccttaccatcatctccatatgagagtgtgaaaataggaacacgt

SEQ ID NO: 152 - DNA, 60 bp, Artificial Sequence
  MammaPrint probe
gctggtggagtagcagatgatattaatactaacaaaaaagaaggaatttcagatgttgtg

SEQ ID NO: 153 - DNA, 60 bp, Artificial Sequence
  MammaPrint probe
tcacccagaaccaatgcggtgtttcttaatgtttgcacaaatttccttaaaaatcaactt

SEQ ID NO: 154 - DNA, 60 bp, Artificial Sequence
  MammaPrint probe
caggacttctctttagtcagggcatgctttattagtgaggagaaaacaattccttagaag

SEQ ID NO: 155 - DNA, 60 bp, Artificial Sequence
  MammaPrint probe
ccctgtgctatcgtaagtttgttttgagcactgcattcactttaaaattctggaggaaca

SEQ ID NO: 156 - DNA, 60 bp, Artificial Sequence
  MammaPrint probe
caacatatttcagttggaaaatttgtatgcagtaatcagccaatgtatttatcggcatcg

SEQ ID NO: 157 - DNA, 60 bp, Artificial Sequence
  MammaPrint probe
cccccattctggaaggttttgttatcttcggaagaaccccaattatgatctctaagtgac

SEQ ID NO: 158 - DNA, 60 bp, Artificial Sequence
  MammaPrint probe
tgtccccagggatcaaacagaagcagccgtgggcaaaatacaatttcatttaacaaattg

SEQ ID NO: 159 - DNA, 60 bp, Artificial Sequence
  MammaPrint probe
aaacagcattatggagttaaaagatttttacaactgggtcttgattttgatgtgagctgg

SEQ ID NO: 160 - DNA, 60 bp, Artificial Sequence
  MammaPrint probe
tccagacgcactgatctttgcaaaggagacttaatttcaaatctgtaattaccatacata

SEQ ID NO: 161 - DNA, 60 bp, Artificial Sequence
  MammaPrint probe
catttggctgtcagaaattataccgagtctactgggtataacatgtctcacttggaaagc

SEQ ID NO: 162 - DNA, 60 bp, Artificial Sequence
  MammaPrint probe
ttaaaggcaaaactgtgctctttattttaaaaaacactgataatcacactgcggtaggtc

SEQ ID NO: 163 - DNA, 60 bp, Artificial Sequence
  MammaPrint probe
aaggtgctgtcatatatcttggaatgaatgacctaaaatcattttaaccattgctactgg

SEQ ID NO: 164 - DNA, 60 bp, Artificial Sequence
  MammaPrint probe
ggatgtaaatcctgagctcaaatctctgttactccattactgtgatttctggctgggtca

SEQ ID NO: 165 - DNA, 60 bp, Artificial Sequence
  MammaPrint probe
cctcaagagtatgtataatttgaagagatactttgtaactatgcttgggtgatattgagc

SEQ ID NO: 166 - DNA, 60 bp, Artificial Sequence
  MammaPrint probe
ttcacagaatagcacaaactacaattaaaactaagcacaaagccattctaagtcattggg

SEQ ID NO: 167 - DNA, 60 bp, Artificial Sequence
  MammaPrint probe
ccagcacataggagagatgagcttcctacagcacaacaaatgtgaatgcagaccaaagaa

SEQ ID NO: 168 - DNA, 60 bp, Artificial Sequence
  MammaPrint probe
gagaaacattgtatattttgcaaaaacaagatgtttgtagctgtttcagagagagtacgg

SEQ ID NO: 169 - DNA, 60 bp, Artificial Sequence
  MammaPrint probe
tactttttggaaaagaataaaccaagaattgattgggcacatcatttcaagaagtccctc

SEQ ID NO: 170 - DNA, 60 bp, Artificial Sequence
  MammaPrint probe
atggagttgctagtaaagcgaagctgattatcctggaaaacactatttacctattttcca

SEQ ID NO: 171 - DNA, 60 bp, Artificial Sequence
  MammaPrint probe
gactgctagtggataataacatcttgactacttaaaaaagggacatattgaaaatcctgg

SEQ ID NO: 172 - DNA, 60 bp, Artificial Sequence
  MammaPrint probe
catgttacctggactggaacagactgtgaatatagcagaaggttccaagaactctggtgt

SEQ ID NO: 173 - DNA, 60 bp, Artificial Sequence
  MammaPrint probe
gagaccaggtgcttcaaaacttaggctcggtagaatcttactcagaagaaaaagcaaaaa

SEQ ID NO: 174 - DNA, 60 bp, Artificial Sequence
  MammaPrint probe
ggattcaacccaaatgatttctcatcaggtgattcttggttgtagcaaagttcatgtgaa

SEQ ID NO: 175 - DNA, 60 bp, Artificial Sequence
  MammaPrint probe
agaactcttgattttgtacatagtcctctggtctatctcatgaaacctcttctcagacca

SEQ ID NO: 176 - DNA, 60 bp, Artificial Sequence
  MammaPrint probe
aattggtaaacatcatgttcctgatgataacccagtagcaaaaacatttgtactgagtgg

SEQ ID NO: 177 - DNA, 60 bp, Artificial Sequence
  MammaPrint probe
catcagtcttgggaaatttgaactttgatcaacttaactaaagaaggaagggtagtaaga

SEQ ID NO: 178 - DNA, 60 bp, Artificial Sequence
  MammaPrint probe
ttagggccctacgtaataggctaattgtactgctcttagaatgtaagcgttcacgaaaat

SEQ ID NO: 179 - DNA, 60 bp, Artificial Sequence
  MammaPrint probe
gagtctttgggataccattaaaaagaagaaaatttcagcctctacaagtcacaacagaag

SEQ ID NO: 180 - DNA, 60 bp, Artificial Sequence
  MammaPrint probe
agagtgtgaaaaataggaacacgtgctctacctccatttagggatttgcttgggatacag

SEQ ID NO: 181 - DNA, 60 bp, Artificial Sequence
  MammaPrint probe
aactttttagggcaaagttaacactgaaagttctagcttaagtgttgaaacttttgtggg

SEQ ID NO: 182 - DNA, 60 bp, Artificial Sequence
  MammaPrint probe
acttagcattttctgcatctccacttggcattagctaaaaccttccatgtcaagattcag

SEQ ID NO: 183 - DNA, 60 bp, Artificial Sequence
  MammaPrint probe
ttttgatgagaatgaatcttggtacttagatgacaacatcaaaacatactctgatcaccc

SEQ ID NO: 184 - DNA, 60 bp, Artificial Sequence
  MammaPrint probe
ttcccttcaatactcctaaaaccaaagaaggatattactaccgtcaagtctttgaacgcc

SEQ ID NO: 185 - DNA, 60 bp, Artificial Sequence
  MammaPrint probe
tcctgtcctgctcattatgccacttccttttaactgccaagaaattttttaaaataaata

SEQ ID NO: 186 - DNA, 60 bp, Artificial Sequence
  MammaPrint probe
caaaaactcagatctatcttaagagtgaccaggaagaggttcattgaaataatcatgcat

SEQ ID NO: 187 - DNA, 60 bp, Artificial Sequence
  MammaPrint probe
catacggttttgtttggaggatggcttctgctgctaaaaatacaaaagtttggaaaccgc

SEQ ID NO: 188 - DNA, 60 bp, Artificial Sequence
  MammaPrint probe
cagagggaccttatttaaacataagtgctgtgacttcggtgaattttcaatttaaggtat

SEQ ID NO: 189 - DNA, 60 bp, Artificial Sequence
  MammaPrint probe
gtttgtgaaactgttaaggtcctttctaaattcctccattgtgagataaggacagtgtca

SEQ ID NO: 190 - DNA, 60 bp, Artificial Sequence
  MammaPrint probe
ttaaccagctgtaaaacacagacctttatcaagagtaggcaaagattttcaggattcata

SEQ ID NO: 191 - DNA, 60 bp, Artificial Sequence
  MammaPrint probe
ggggatgaatagaaaacctgtaagctttgatgttctggttacttctagtaaattcctgtc

SEQ ID NO: 192 - DNA, 60 bp, Artificial Sequence
  MammaPrint probe
gtgataaagtacctgatccaaatgttatgagaatactggacgagaattgaacgaaattga

SEQ ID NO: 193 - DNA, 60 bp, Artificial Sequence
  MammaPrint probe
tgcagcagtactactgtcaacatagtgtaaatggttctcaaaagcttaccagtgtggact

SEQ ID NO: 194 - DNA, 60 bp, Artificial Sequence
  MammaPrint probe
gcatgagtcacaattacaaagttttgagcggttttgtaatttgacatttaggaaagtctc

SEQ ID NO: 195 - DNA, 60 bp, Artificial Sequence
  MammaPrint probe
ttattcgaagacacagaagttgggcaagtcaaatgttgtgtcgtcagttgtgcatccgtt

SEQ ID NO: 196 - DNA, 60 bp, Artificial Sequence
  MammaPrint probe
tgtactggcaggctcgttttacctgattctagaatatttaagaatctaaaaataaagggc

SEQ ID NO: 197 - DNA, 60 bp, Artificial Sequence
  MammaPrint probe
gtggcctataacttacttgtcaacaactgtgaacattttgtgacattgcttcgctatgga

SEQ ID NO: 198 - DNA, 60 bp, Artificial Sequence
  MammaPrint probe
ccaggacgccactcatttcatctcatttaagggaaaaatatatatctatctatttgagga

SEQ ID NO: 199 - DNA, 60 bp, Artificial Sequence
  MammaPrint probe
cggagcgcagggtacttggcgtataataagccatcaataatttatgggtgaaattgagag

SEQ ID NO: 200 - DNA, 60 bp, Artificial Sequence
  MammaPrint probe
cagagctacaactaggaaaattagagtggtagtagtcacttatttaagaattcattcagg

SEQ ID NO: 201 - DNA, 60 bp, Artificial Sequence
  MammaPrint probe
ttggtagttaaccctaactacttgctcgaagattgagatagtgaaagtaactgaccagag

SEQ ID NO: 202 - DNA, 60 bp, Artificial Sequence
  MammaPrint probe
gcgtgagcatgtcagtattctagtccagtatttgccagtttccaagtaaaagcttttgtg

SEQ ID NO: 203 - DNA, 60 bp, Artificial Sequence
  MammaPrint probe
gctgtgccattcaatgtttgatgcataattggaccttgaatcgataagtgtaaatacagc

SEQ ID NO: 204 - DNA, 60 bp, Artificial Sequence
  MammaPrint probe
ccaagaaggaaaatgtcaaaattagtgatgagggaatagcttatcttgttaaagtgtcag

SEQ ID NO: 205 - DNA, 60 bp, Artificial Sequence
  MammaPrint probe
tgctttaagtgaatggcagtcccttgtcttattcagaatataaaattcagtctgaatggc

SEQ ID NO: 206 - DNA, 60 bp, Artificial Sequence
  MammaPrint probe
aggttggcagtaaggcagggtcccatttctcactgagaagattgtgaatatttccatatg

SEQ ID NO: 207 - DNA, 60 bp, Artificial Sequence
  MammaPrint probe
gtgcaaatagaattagcagtaagaagctactctagctaatttgccatttcacttaaatgg

SEQ ID NO: 208 - DNA, 60 bp, Artificial Sequence
  MammaPrint probe
gatacagcctacataaagactgttatgatcgctttgattttaaagttcattggaactacc

SEQ ID NO: 209 - DNA, 60 bp, Artificial Sequence
  MammaPrint probe
caacatttacattgtaattcaatagacgctactactacaaaggagctttattcttccagc

SEQ ID NO: 210 - DNA, 60 bp, Artificial Sequence
  MammaPrint probe
caacagtattgcgttgtcagactaggaaagctaaacgaacaaaatggttttagttttgct

SEQ ID NO: 211 - DNA, 60 bp, Artificial Sequence
  MammaPrint probe
ctggttgtccaactaccatatgaagctagaaaatgcacaaacgatattccttatctgtaa

SEQ ID NO: 212 - DNA, 60 bp, Artificial Sequence
  MammaPrint probe
ggcatcagggatcacatcactcttaacggctgttacttaaacaactattttttggtttgg

SEQ ID NO: 213 - DNA, 60 bp, Artificial Sequence
  MammaPrint probe
tgaaaatgtatttgtagtcacggactttcaggattctgtctttaatgacctctacaaggc

SEQ ID NO: 214 - DNA, 60 bp, Artificial Sequence
  MammaPrint probe
agaccaggtctctattttgaggaagaaataccgagacattgagcgactttgaggaatccg

SEQ ID NO: 215 - DNA, 60 bp, Artificial Sequence
  MammaPrint probe
aagtcatgacacagtattcgctctttttctgaatgtttacatagagattcatcactgcag

SEQ ID NO: 216 - DNA, 60 bp, Artificial Sequence
  MammaPrint probe
cagtaagtacgggaaaaaatgtttactaacttcctcagagattcgtgatacgcgtttctc

SEQ ID NO: 217 - DNA, 60 bp, Artificial Sequence
  MammaPrint probe
ctttgaatggacataaaaattctgcttgttaagaacaagttgagctctggtaactgatct

SEQ ID NO: 218 - DNA, 60 bp, Artificial Sequence
  MammaPrint probe
tgactgatgtgtctgaaaatgctaaggatcttattcgaaggctcatttgtagcagagaac

SEQ ID NO: 219 - DNA, 60 bp, Artificial Sequence
  MammaPrint probe
ctctgaaagaagaagttcaaaagctggatgatctttaccaacaaaaaattaaggaagcag

SEQ ID NO: 220 - DNA, 60 bp, Artificial Sequence
  MammaPrint probe
atctgtggttattcgaacctttattactagtgacttcatgactggtatacctgcaacacc

SEQ ID NO: 221 - DNA, 60 bp, Artificial Sequence
  MammaPrint probe
tccaccccaggacgccactcatttcatctcatttaagggaaaaatatatatctatctatt

SEQ ID NO: 222 - DNA, 60 bp, Artificial Sequence
  MammaPrint probe
ggccctctcttctcacctttgttttttgttggagtgtttctaataaacttggattctcta

SEQ ID NO: 223 - DNA, 60 bp, Artificial Sequence
  MammaPrint probe
ctgggttgatacctgaaagaatcctgtcttatttggtctccataatcctttgaatggaaa

SEQ ID NO: 224 - DNA, 60 bp, Artificial Sequence
  MammaPrint probe
agtaccctgatatactgaattttgtggatgatttggaacctttagacaaagctagtaaag

SEQ ID NO: 225 - DNA, 60 bp, Artificial Sequence
  MammaPrint probe
aagactttcttactgacctgaataacttcagaaccacattcatgcaagcaataaaggaga

SEQ ID NO: 226 - DNA, 60 bp, Artificial Sequence
  MammaPrint probe
tttagtggtccgttgcctctgaagatgtaaacaaacaaatacactatttctgggaacatt

SEQ ID NO: 227 - DNA, 60 bp, Artificial Sequence
  MammaPrint probe
atagaatatgtatatgtattctttgtctaccaactaccaaagaaacaaatactcctcagt

SEQ ID NO: 228 - DNA, 60 bp, Artificial Sequence
  MammaPrint probe
acattgcttacttaaaagctacatagccctatcgaaatgcgaggattaatgctttaatgc

SEQ ID NO: 229 - DNA, 60 bp, Artificial Sequence
  MammaPrint probe
accataaggcaattgagcacataacgaaaaatgatgcaataagaatgtatgcactctctt

SEQ ID NO: 230 - DNA, 60 bp, Artificial Sequence
  MammaPrint probe
cagcctttcctcatgtcaacacagttcacaatatagttttcaaagtacagtttaaaactc

SEQ ID NO: 231 - DNA, 60 bp, Artificial Sequence
  MammaPrint probe
cctccccaaaataattagtaactggttgttctacttggtaatttgacaccctgttaataa